(19) United States
(12) Patent Application Publication
(10) Pub. No.: US 2018/0353765 A1
(43) Pub. Date: Dec. 13, 2018

(54) MAGNETOGENETICS AND USES THEREOF

(71) Applicants: Shengjia Zhang, Trondheim (NO); TSINGHUA UNIVERSITY, Beijing (CN)

(72) Inventors: Shengjia Zhang, Trondheim (NO); Xiaoyang Long, Chenzhou (US)

(21) Appl. No.: 15/756,554

(22) PCT Filed: Aug. 31, 2015
(86) PCT No.: PCT/CN2015/000622

§ 371 Date: Feb. 28, 2018

(51) Int. Cl. A61N 2/00; A61K 38/17; A61K 48/00; A61P 25/00

(52) U.S. Cl. CPC A61N 2/002; A61K 38/177; A61P 25/00; A61K 48/0058; A61K 48/0083

(57) ABSTRACT

Provided is a non-invasive method of modulating the activity of a cell, comprising the steps of delivering a MAR gene into said cell and applying a magnetic stimulation to said cell. The medical use of magnetogenetics in treating diseases is also provided.

### FIELD OF THE INVENTION

The present invention relates to the field of magnetogenetics. In particular, the present invention relates to a magnetoreceptor which responds to external magnetic stimulation, and non-invasive methods of modulating neuronal activity, perturbing biological processes and treating diseases.

### BACKGROUND

The complex neural microcircuits are the essential building blocks of how the brain works but they are entangled with interdependent different cell types, interconnected wiring diagrams and internetworked complicated connectome in vivo (Bargmann et al., 2014; Luo et al., 2008). Understanding how neural circuits respond to external stimuli, generate electric firing patterns, process information, compute coding and orchestrate behavior has, therefore, remained a great challenge for neuroscientists (Harris and Mrsic-Flogel, 2013; Huang and Zeng, 2013). With continuous development and maturation, many neurotechnological toolboxes including optogenetics (Zhang et al., 2011), chemogenetics (Lerchner et al., 2007; Vardy et al., 2015), deep-brain stimulation (Wichmann and Delong, 2006) and functional magnetic resonance imaging (fMRI) (Kwong et al., 1992; Logothetis, 2008) have been proven to play an important role in dissecting, perturbing and modulating interconnected neural microcircuits in the healthy and diseased brain. Among those well-developed neurotechnological toolboxes, both classical deep brain stimulation and modern optogenetics make it possible to map, monitor and manipulate physiological and dysfunctional neural microcircuit activity (Gradinaru et al., 2009; Logothetis, 2008). However, they all have their own limitations or drawbacks. The classical deep brain stimulation has been successfully used to treat Parkinson's disease and other neurological disorders but its limitations are the necessity of surgical implant of an electrical wire, the lack of spatial selectivity or specificity, as well as its contradictory effect of low-frequency and high-frequency stimulation on neuronal excitation or inhibition, respectively (Kringelbach et al., 2007). Even though the most popular optogenetics could spatiotemporally activate or deactivate neural activity with a millisecond precision (Bi et al., 2006; Boyden et al., 2005; Han and Boyden, 2007; Li et al., 2005; Zhang et al., 2007) and has rapidly transformed neuroscience, the side effects from opsin expression patterns, laser-induced heating, abnormal ions distribution caused by overexpressed pumps or channels, and/or undesired network homeostasis can make experimental interpretation very difficult (Häusser, 2014). Both optogenetics and deep-brain stimulation have been used to invasively manipulate the neuronal activity of a specific sub-region in the intact mammalian brain through a permanently implanted electric wire or optical fiber during the chronic surgery (Grosenick et al., 2015; Logothetis, 2008; Okun, 2012). As a result, there has been a high demand on a new generation of exclusively non-invasive neuroperturbation and neuromodulation toolboxes for the whole brain at both microcircuit and macrocircuit levels.

### SUMMARY OF THE INVENTION

In one aspect the present invention provides a method of modulating the activity of a cell, comprising the steps of delivering a MAR gene into said cell and providing a magnetic stimulation to said cell.

The cell may be treated in vivo, e.g. in an animal such as a human, or be treated in vitro, e.g. in a culture dish.

Particularly, the cell may be a neuron cell, muscle cell or stem cell. In an embodiment, the cell may be in a subject, e.g., a primate such as a human, or a rodent such as a mouse, rat or rabbit.

In an embodiment, the MAR gene may be delivered to a target location, e.g., a particular cell type such as a neuron or a particular region of a healthy or diseased organ, via a vector comprising a cell type specific promoter or region specific promoter. The vector may comprise a lentivirus, a retrovirus or adeno-associated virus or a plasmid.

In another aspect the present invention provides a method of treating a neurodegenerative disease in a subject, comprising the steps of delivering a MAR gene into said subject via a vector and providing a magnetic stimulation to said subject.

The neurodegenerative diseases include, but not limited to, Alzheimer's disease, Parkinson's disease, Prion disease, Motor neuron diseases, Huntington's disease, Spinocerebellar ataxia, and Spinal muscular atrophy.

In an embodiment, the MAR gene is targeted to one or more diseased regions by a vector which comprises a cell type specific promoter or region specific promoter.

In an embodiment, the MAR gene is delivered by implanting a MAR-expressing cell into said subject.

In another aspect the present invention provides a method of repairing spinal cord injury in a subject, comprising the steps of delivering a MAR gene into an injured target region via a vector and providing a magnetic stimulation to said subject.

In another aspect the present invention provides a method for targeted magnetogenetic treatment of retina-degenerative diseases including blindness and retinitis pigmentosa, comprising the steps of delivering a MAR gene into a target region via a vector and providing a magnetic stimulation to said region.

In another aspect the present invention provides a method for targeted cardiac treatment, comprising the steps of delivering a MAR gene into a target region in the heart via a vector and providing a magnetic stimulation to said region.

In another aspect the present invention provides a method for treating Sjögren's syndrome in a subject, comprising the steps of delivering a MAR gene into the subject via a vector and providing a magnetic stimulation to said subject.

In another aspect the present invention provides a vector for delivering magnetoreceptor MAR comprising a nucleic acid sequence that codes for MAR protein and a cell type specific promoter or region specific promoter.

In an embodiment, the vector comprises a virus, e.g., a lentivirus, a retrovirus or adeno-associated virus, or a plasmid.

In another aspect the present invention provides a transgenic animal which expresses an exogenous MAR gene and can respond to external magnetic stimulation. In some embodiments, the animal is a fly, worm, zebrafish, mouse, rat or marmoset.

In another aspect the present invention provides a method of diagnostic or therapeutic magnetic resonance imaging in combination with MAR-dependent magnetic stimulation, comprising: monitoring a neural reaction with magnetic resonance imaging and modifying a targeted brain region expressing MAR and stimulating the brain with external magnetic field to activate neuronal activity.

In another aspect the present invention provides a method for targeted magnetogenetic treatment of cardiac diseases including irregular heart rhythm, comprising the steps of delivering a MAR gene into a target region in the heart via a vector and providing a magnetic stimulation to heart muscle.

In another aspect the present invention provides a pharmaceutical composition for treating a subject, comprising: a vector comprising a MAR gene, or a MAR-expressing cell; and a pharmaceutically acceptable carrier.

In another aspect the present invention provides a method of deep brain stimulation with a magnetic field for treating a disease such as Parkinson's disease, chronic pain, major depression, Tourette syndrome or epilepsy, comprising the steps of delivering a vector comprising a MAR gene into a target diseased region and providing a magnetic stimulation to the region.

In another aspect the present invention provides a method of non-invasive magnetic stimulation of a MAR-targeted brain region, comprising the steps of delivering a vector comprising a MAR gene into the MAR-targeted brain region and providing a magnetic stimulation to the region.

In another aspect the present invention provides a method of treating a subject, comprising the steps of delivering a MAR gene to a target region in the subject and providing a magnetic stimulation to the region. The subject may be healthy or have a disease or injury.

In another aspect the present invention provides a method of magnetically inhibiting a target region in a subject, comprising the steps of molecular engineering of a MAR gene and/or a magnetoreceptor family member in the subject and providing a magnetic stimulation to the region.

In another aspect the present invention provides a method of generating a secondary messenger in a cell by expressing a MAR protein and a MAR-interacting receptor, comprising the steps of delivering a vector comprising a MAR gene into the cell and providing a magnetic stimulation to the cell, wherein the expression of the MAR protein provides for production of a secondary messenger and/or perturbation of signal transduction pathways in the cell.

In another aspect the present invention provides a fusion protein comprising a MAR protein coupled to another functional protein, wherein the other functional protein is a fluorescent protein, such as mCherry, GFP, YFP, or CFP.

In some embodiments, the other functional protein has a PDZ or AIS domain.

In some embodiments, the other functional protein targets a subcellular region.

In another aspect the present invention provides a method of control of memory function for disrupting the formation and recall of memories, comprising the steps of expressing magnetoreceptor MAR in brain areas such as the hippocampus, the amygdala and/or the cingulate cortex and stimulating the brain with external magnetic field.

In another aspect the present invention provides a method for treating addiction such as drug addiction and alcohol addiction in a subject, comprising the steps of expressing magnetoreceptor MAR in brain areas such as the nucleus accumbens and stimulating the brain with external magnetic field.

In another aspect the present invention provides a method of control of excitation and neurogenesis in neural stem/progenitor cells, comprising the steps of expressing magnetoreceptor MAR in stem cells and activating with external magnetic field to enhance neurogenesis.

In another aspect the present invention provides a method of magnetogenetic control of endothelial cells by transporting MAR across the vascular barrier into tissues such as the brain and the lung, and controlling vascular properties such as vascular tone, arterial diameter, and vascular growth by external magnetic field applied.

In another aspect the present invention provides a nucleic acid sequence comprising a gene for MAR and an inducible promoter.

In some embodiments, the inducible promoter is inducible by a trans-acting factor which can respond to an administered drug.

### BRIEF DESCRIPTION OF THE DRAWINGS

The figures depict non-limiting exemplary embodiments of the technology disclosed herein and are provided to aid the reader in understanding the disclosure.

FIG. 1A to 1F are graphs showing the magnetogenetic activation of HEK-293 cells by remote magnetic stimulation.

(A) Membrane depolarization induced by electric coils. Left, schematic of magnetic stimulation of MAR-GCaMP6s co-transfected HEK-293 cells by a pair of electrical coils. Middle and right, heat map showing change of fluorescence intensity (ΔF/F0) before and after magnetic field stimulation. Scale bar, 50 μm.

(B) Activation of HEK-293 cells with magnetic field generated by a pair of bar magnets. Middle and right, color map of fluorescence change of GCaMP6s triggered by external magnetic field. Scale bar, 50 μm.

(C) Population activity showed increased fluorescence intensity only in MAR positive cells after magnetic stimulation while fluorescence intensity of control group remained at the base level. Solid lines, mean; shaded gray areas, s.e.m. Blue bar indicates field on. Inset was magnified view showing onset latency of about 13 seconds after stimulus onset. Dashed line indicated response onset when ΔF/F0 was 10 folds of the standard deviation of the baseline fluctuation.

FIG. 2A to 2E are graphs showing that MAR enables magnetic-control of neuronal activity.

(A) Schematic of calcium imaging with hippocampal neurons cultured in Tyrode's solution.

(B) Confocal imaging showing co-localization of GCaMP6s and MAR.

(C) Time course of average peak ΔF/F0 as a function of time (Solid lines indicate the mean value and shaded gray areas indicate s.e.m.). Calcium transients were only observed in MAR-transfected group. Orange, MAR group, n=42; black, control group, n=40. Blue bar indicates field-on.

(D) Distribution of peak ΔF/F0, onset latency and duration. Each gray dot represents result from a single neuron, while solid dots indicate mean value. Mean peak ΔF/F0 was 50.5±7.0%; mean onset latency was 7.8±0.8 s; mean duration of MAR-evoked calcium transients was 11.1±0.9 s. Error bar, s.e.m.

(E) Representative traces from two neurons co-expressing MAR and GCaMP6s. MAR was able to trigger calcium spikes repeatedly with the external magnetic field. Blue bar indicates field-on.

FIG. 3A to 3D are graphs showing magnetogenetic control of neuronal activity in a direction-selective and polarity-oriented manner.

(A) Direction-selective magnetic activation of calcium influx. Schematic of two-directional magnetic stimulation set-up (see also FIG. 51).

(B) Sample traces of fluorescence intensity of three neurons in response to magnetic fields of different directions in X-Y plane. Green arrow, direction of magnetic field in X-axis. Orange arrow, direction of magnetic field in Y-axis. Green bar, field on in X-direction; orange bar, field on in Y-direction. Left, a representative neuron exhibited a large calcium peak when the magnetic field was turned on to X-axis while only a small peak was observed when the magnetic field was switched to Y-axis. Middle, an example neuron responded only to the magnetic stimulation along Y-axis. Right, representative trace showing calcium spikes to magnetic field along both X-axis and Y-axis.

(C) On-response and off-response patterns of neuronal activity. Schematic showing switch-on and switch-off of magnetic field induced on-response and off-response patterns of neuronal activity.

(D) Fluorescence traces shown were three representative neurons with different response patterns. Upper, a neuron exhibiting calcium transient when the magnetic field was turned on (on-response), but not when it was turned off (off-response). Middle, a neuron exhibiting off-response but not on-response. Lower, a neuron exhibiting both on-response and off-response. Blue bar, field-on; orange bar, field-off.

FIG. 4A to 4E are graphs showing the neuronal spiking activity driven by the magnetic field via MAR.

(A) Experiment scheme of whole-cell patch-clamp recording. Magnetic stimulation was achieved through a pair of hand-held magnets.

(B) Confocal imaging of a typical MAR-p2A-mCherry expressing neuron. Scale bar, 30 μm.

(C) Current-clamp recording showing changes of membrane potential to magnetic stimulation. Three example neurons exhibited membrane depolarization and increasing firing rate to the onset of magnetic field. Scale bar, 20 s, 50 mV.

(D) MAR triggered action potentials displayed on-response and off-response firing patterns. Voltage traces of three representative neurons showed distinct firing patterns in response to magnetic field-on and field-off. Upper, the neuron only fired action potentials to the onset of magnetic field. To the opposite, the neuron shown in middle panel mainly responded to the removal of magnet. While another group showed typical firing pattern (lower panel) that both switch-on and switch-off of magnetic field elicited action potentials. Blue bar, field-on; orange bar, field-off.

(E) Magnetic field induced significant increase in number of action potentials with mean onset latency of 5.3±1.1 s and average duration of 8.5±1.5 s when compared to spontaneous firing rate (13.2±4.2 spikes versus 1.0±0.5 spikes; n=19;**, P<0.01, paired t-test). Error bar, s.e.m.

FIG. 5A to 5F are graphs showing the magnetogenetic control of behavioral responses in C. elegans.

(A) Epifluorescence image of MAR expression in the body wall of C. elegans under the promoter myo-3.

(B) Simultaneous contraction of body muscle when magnetic field was applied under white field illumination. Asterisks indicate the head and tail of C. elegans. Left, body relaxation just before magnetic field was on; right, body contraction after the magnetic field was switched on.

(C) Body length was measured with 1 second interval at 10 s before and 50 seconds after magnetic field was turned on and also at 20 s after magnetic field was turned off. Relative body length was calculated by dividing the length measured to the average body length before stimulus onset. Orange trace showing reduction of body length to 94% of the initial length while N2 wild type showed no obvious change of body length by magnetic stimulation (myo-3, n=24; N2, n=20).

(D) MAR was selectively expressed in gentle touch receptor neurons under mec-4 promoter. Shown is a PLM neuron. Scale bar, 5 μm.

(E) Withdrawal behavior was elicited in the mec-4 transgenic animal when magnetic field was on. Animal positions from 3 frames after stimulus onset at 0 s, 3 s, and 6 s were shown by white, orange, blue outline, respectively.

(F) Percentage of responding transgenic animals in five consecutive trails with obvious withdrawal or forwarding behavior (with travelling distance of at least ¼ body length) by magnetic stimulation. The fraction of zdEx22 transgenic C. elegans was 86% in the first trail and showed gradual habituation when tested repeatedly.

FIG. S1A to S1C are graphs showing calcium influx by repetitive magnetic stimulation in cultured hippocampal neuron.

(A) Heat map showing change of fluorescence intensity of a representative neuron by repetitive magnetic stimulation. Scale bar, 30 μm.

(B) Trace of relative fluorescence change in A by repetitive magnetic stimulation. Blue bars, field-on.

(C) Spontaneous fluorescence intensity of a representative neuron was normalized to 1.0 at t=0. Normalized fluorescence was fitted using mono-exponential equation. Traces were then corrected with the time constant derived for photobleaching effect.

FIG. S2 shows the fraction distribution of direction-dependent activation and On-Off response pattern of neuronal activity by magnetic stimulation. Quantification of the angle between the axonal orientation of the responsive neuron and the corresponding stimulating direction of the magnetic field. No significant difference was found between the X-responsive, Y-responsive and both X- and Y-responsive groups (P>0.3, ANOVA test, n=9, 6 and 4, respectively). Error bar, s.d.

FIG. S3 shows the summary of angle distribution between axonal orientation of the responsive neurons and direction of field.

FIG. S4A to S4D are graphs showing magnetic field evoked currents and intrinsic properties of MAR-transfected neurons.

(A-B) Representative traces showing inward (traces#1-3) and outward (traces#4-6) currents by clamping neurons at −70 mV and 0 mV, respectively.

(C) Comparison between magnetic field-evoked inward currents and spontaneous currents. Mean inward peak current evoked by magnetic stimulation was 279.6±45.2 pA versus 33.3±17.8 pA of spontaneous current (***, P<0.001, paired t-test, n=13). The average number of events evoked was 9.3±3.95 versus 0.46±0.24 (*, P<0.05, paired t-test).

(D) Comparison of intrinsic properties between MAR-positive and MAR-negative neurons. Resting membrane potential in MAR expressing neurons (−53.4±3.2 mV, n=14) was not significantly different from neurons not expressing MAR (−52.3±2.4 mV, n=10). P>0.4, student t-test. Membrane resistance was measured under voltage-clamp mode by injecting a 10 mV voltage step. No statistical difference was found between MAR-positive and MAR-negative neurons (130.6±18.9 MΩ versus 119.8±12.9 MO). P>0.3, student t-test.

FIGS. S5A and S5B are graphs showing epifluorescence image of MAR-expressed muscle cells and mechanosensory neurons.

(A) Epifluorescence photos showing MAR-localization in the body wall muscle cells indicated by the arrows under the promoter myo-3 (transgene zdEx12).

(B) Magnified view of MAR expression in six mechanosensory neurons. Left, arrows indicate three neurons (AVM, ALMR, PLMR). Right, fluorescent images of the other three neurons (PVM, ALML, PLML).

### DETAILED DESCRIPTION OF THE INVENTION

It is to be appreciated that certain aspects, modes, embodiments, variations and features of the technology disclosed herein are described below in various levels of detail in order to provide a substantial understanding of the technology, and shall not be construed to limit the protection scope as defined in the appended claims.

Summary

Current neuromodulation techniques such as optogenetics and deep-brain stimulation are transforming basic and translational neuroscience. These two neuromodulation approaches are, however, invasive since surgical implantation of an optical fiber or wire electrode is required. Here, we have invented non-invasive magnetogenetics that combines the genetic targeting of a magnetoreceptor with remote magnetic stimulation. The non-invasive activation of neurons was achieved by neuronal expression of an exogenous magnetoreceptor that was discovered before and evolutionarily highly conserved. In HEK-293 cells and cultured hippocampal neurons expressing this magnetorecepter, application of an external magnetic field resulted in membrane depolarization and calcium influx in a reproducible and reversible manner, as indicated by the ultrasensitive fluorescent calcium indicator GCaMP6s. Moreover, the magnetogenetic control of neuronal activity might be dependent on the direction of the magnetic field and exhibits on-response and off-response patterns for the external magnetic field applied. The activation of this magnetoreceptor can depolarize neurons and elicit trains of action potentials, which can be triggered repetitively with a remote magnetic field in whole-cell patch-clamp recording. In transgenic Caenorhabditis elegans expressing this magnetoreceptor in myo-3-specific muscle cells or mec-4-specific neurons, application of the external magnetic field triggered muscle contraction and withdrawal behavior of the worms, indicative of magnet-dependent activation of muscle cells and touch-receptor neurons, respectively. The advantages of magnetogenetics over optogenetics are its exclusive non-invasiveness, deep penetration, unlimited accessibility, spatial uniformity and relative safety. Like optogenetics that went through a decade-long improvements, magnetogenetics, with continuous modification and maturation, will reshape the current landscape of neuromodulation toolboxes and will have a broad range of applications to basic and translational neuroscience as well as other biological sciences. We envision a new age of magnetogenetics is coming.

MAR

Although the mechanism for magnetoreception has remained elusive, it has been proposed that to maximize the ability of animals to sense the geomagnetic fields, cryptochrome (Cry), a well-known magnetosensor needs to interact with another magnetoreceptor. There are many candidate interacting partners for Cry to form a stable complex, and one of which is the bacterial IscA1, an iron-sulfur protein (Cózar-Castellano et al., 2004) that might bind to Cry to form a stable complex to regulate iron-cluster assembly within cells. Interestingly, IscA1 is very conserved among butterfly, rat, mouse, pigeon, drosophila and human with very high homology. We reason that those extremely conserved IscA1 might function as a universal magnetoreceptor (termed as MAR) and induce neuronal activity after stimulation with the external magnetic field. Since pigeon has the strongest magnetic sensing system, we therefore expressed codon-optimized pigeon magnetoreceptor version for mouse, rat, marmoset and human (MAR, named thereafter in our study).

The terms “IscA1” and “MAR” are used interchangeably herein and refer to the same protein which is highly conservative across organisms. The IscA1 protein (or MAR protein) in pigeon contains 133 amino acids. The term“IscA1” or“MAR” encompasses any homologues of IscA1 in different organisms which share a high sequence identity, e.g., more than 70%, 75%, 80%, 85%, 90%, 95%, or even 98%, with pigeon IscA1 and possess substantially the same biological function of responding to magnetic stimulation.

As used herein, the term “MAR protein” encompasses the full protein, or a variant thereof which maintains substantially the same biological functions as the native full MAR.

It will be appreciated by a skilled artisan that a native MAR protein from different organisms including bacteria, butterfly, pigeon, mouse, rat, marmoset, monkey or human, or a functional variant thereof may be used in the present invention.

As used herein, the term“protein” is interchangeable with the term “polypeptide” or “peptide”.

The term “MAR protein” encompasses variants of the naturally occurring protein. Preferably, the variant has a sequence identity of at least 75%, preferably 80%, more preferably 85%, or even 90%, 95% or 98% with the naturally occurring protein. The sequence identity may be determined using standard techniques known in the art, e.g. BLAST.

In a preferred embodiment, the MAR protein of the present invention is a variant (also termed as a functional variant), as compared to native MAR, but maintains substantially the same biological functions as the native MAR. That is, the variant MARprotein contains substitutions, deletions or insertions of one or several amino acids, e.g, of 3, 5, 8, 10, 12 or 15 amino acids, in the native MAR sequence.

These variants ordinarily are prepared by site specific mutagenesis of nucleotides in the encoding DNA or other techniques well known in the art, to produce DNA encoding the variant, and thereafter expressing the DNA in recombinant cell culture. The variants typically exhibit the same qualitative biological activity as the naturally occurring analogue.

The MAR protein of the present invention can incorporate un-natural amino acids as well as natural amino acids. The unnatural amino acids can be used to enhance ion selectivity, stability, compatibility, or to lower toxicity.

An aspect of the present invention is a fusion protein comprising MAR protein. It is well known in the art that fusion proteins can be made that will create a single protein with the combined activities of several proteins. Desirable properties such as elongated half-life might be achieved by the fusion protein.

One embodiment of a fusion protein comprising MAR protein is a fusion protein that targets sub-cellular regions of the cell. The fusion proteins can target, for instance, axons, dendrites, and synapses of neurons. In one preferred embodiment, a PDZ (PSD-95, DIg and ZO-1) domain is fused to MAR which targets dendrites. In another preferred embodiment, Axon initial segment (AIS) domain is fused to MAR which targets axons.

Another aspect of the present invention provides nucleic acid sequences which code for the MAR protein. It would be understood by a skilled artisan that the MAR protein can be coded for by various nucleic acids. Since many amino acids are represented by more than one codon, there is not a unique nucleic acid sequence that codes for a given protein. It is well understood by a skilled artisan how to make a nucleic acid that can code for a MAR protein by knowing the amino acid sequence of the protein. A nucleic acid sequence that codes for a polypeptide or protein is the “gene” of that polypeptide or protein. A gene can be RNA, DNA, or other nucleic acid than will code for the polypeptide or protein. Other sequences which do not substantially alter the amino acid sequence of a MAR protein can be added in the MAR gene, such as introns. The term“MAR gene” refers to a nucleic acid sequence that codes for a MAR protein (see, e.g., SEQ ID NOs:1-6).

SEQ ID NO: 1, natural MAR gene in fruit fly.
ATGGCGACACGTGTGGTGGCAACGGCGACAGTGCGGGCGGTGAAAGGCCG
GAAGTTAATCCCGACGCGGGCCGCTCTGACTCTGACACCCGCGGCGGTGC
TACGCATCAAGACGCTTCTGCAGGACAAGCCGGACATGGTTGGCCTAAAG
GTGGGCGTACGGCAGCGAGGATGCAATGGTCTGTCCTACACGCTGGACTA
TGCCAGCCAAAAAGACAAGTTGGATGAGGAGGTGGTCCAGGATGGCGTCA
AGGTCTTCATCGACAAGAAAGCGCAGTTGTCGCTGCTGGGTACCGAGATG
GACTTTGTGGAATCGAAGCTGTCCAGCGAGTTCGTGTTTAACAATCCGAA
CATTAAGGGCACATGCGGCTGCGGCGAATCGTTCAGCATGTAA
SEQ ID NO: 2, natural MAR gene in butterfly.
ATGTCTACTAAAACTATAGCAAGTGCAACTGTTAGGGCAGTAAAAAAGCG
TCTGCTACCATCCAGAGCTGCTCTAGTTTTGACTTCTTCAGCCGTAAATA
AAGTTAAGGAAATAATGGCCAAGGAAGAAGGCAAGGGTTATATAGGATTG
AAAGTTGGTGTGCGGCAAAGAGGTTGCAATGGATTGTCATATACCTTAGA
TTATGCAACATCAAAAGGGAAACTTGACGAAGAAGTAAAACAGGATGGAG
TCACTATAATTATTGACAAAAAAGCACAGTTGACCTTGTTGGGTACTGAA
ATGGATTTTGTCGAAGATAAGCTGTCAGCGGAATTTGTGTTTAACAATCC
GAATATAAAAGGCACTTGTGGATGTGGAGAATCTTTCAGTATATAA
SEQ ID NO: 3, natural MAR gene in pigeon.
ATGGCTTCTTCTGCTTCTTCTGTTGTTCGTGCTACCGTTCGTGCTGTTTC
TAAACGTAAAATCCAGGCTACCCGTGCTGCTCTGACCCTGACCCCGTCTG
CTGTTCAGAAAATCAAAGAACTGCTGAAAGACAAACCGGAACACGTTGGT
GTTAAAGTTGGTGTTCGTACCCGTGGTTGCAACGGTCTGTCTTACACCCT
GGAATACACCAAATCTAAAGGTGACTCTGACGAAGAAGTTGTTCAGGACG
GTGTTCGTGTTTTCATCGAAAAAAAAGCTCAGCTGACCCTGCTGGGTACT
GAAATGGACTACGTTGAAGACAAACTGTCTTCTGAATTCGTTTTCAACAA
CCCGAACATCAAAGGTACTTGCGGTTGCGGTGAATCTTTCAACATCTAA
SEQ ID NO: 4, natural MAR gene in mouse.
ATGTCGGCGTCGTTGGTCCGCGCCACCGTGCGGGCTGTGAGCAAGAGAAA
ACTGCAGCCCACGCGGGCGGCCCTCACACTGACCCCCTCTGCTGTAAACA
AGATAAAACAACTTCTTAAAGACAAACCTGAGCATGTGGGTCTGAAAGTT
GGCGTGCGAACCAGGGGCTGTAATGGCCTCTCTTACAGCCTGGAGTACAC
AAAGACAAAAGGAGATTCTGATGAAGAAGTTATTCAAGATGGAGTCCGAG
TGTTCATCGAGAAGAAAGCACAGCTAACCCTGTTAGGAACAGAGATGGAC
TATGTGGAAGACAAACTGTCCAGTGAGTTTGTGTTCAATAACCCCAACAT
CAAGGGAACCTGTGGCTGCGGTGAGAGCTTTCACGTGTGA
SEQ ID NO: 5, natural MAR gene in rat.
ATGTCGGCGTCGTTGGTCCGCGCCACCGTGCGGGCCGTGAGCAAGAGAAA
ACTGCAACCCACGCGGGCGGCGCTCACGCTGACCCCCTCTGCTGTGAACA
AGATAAAACAACTTCTTAAAGACAAGCCTGAGCATGTGGGTCTGAAAGTG
GGTGTGCGGACCAGGGGCTGTAACGGCCTCTCTTACAGCCTGGAGTATAC
AAAGACAAAAGGAGATGCTGATGAAGAAGTTATTCAAGACGGAGTCCGAG
TGTTCATCGAGAAGAAAGCCCAGCTAACCCTGTTAGGCACAGAGATGGAC
TATGTGGAAGACAAACTGTCCAGTGAGTTTGTGTTCAACAACCCCAACAT
CAAGGGAACCTGTGGCTGCGGTGAAAGCTTTAACGTCTGA
SEQ ID NO: 6, natural MAR gene in human.
ATGTCGGCTTCCTTAGTCCGGGCAACTGTCCGGGCTGTGAGCAAGAGGAA
GCTGCAGCCCACCCGGGCAGCCCTCACCCTGACACCTTCAGCAGTAAACA
AGATAAAACAACTTCTTAAAGATAAGCCTGAGCATGTAGGTGTAAAAGTT
GGTGTCCGAACCAGGGGCTGTAATGGCCTTTCTTATACTCTAGAATATAC
AAAGACAAAAGGAGATTCTGATGAAGAAGTTATTCAAGATGGAGTCAGAG
TATTCATCGAAAAGAAAGCACAGCTAACACTTTTAGGAACAGAAATGGAC
TATGTTGAAGACAAATTATCCAGTGAGTTTGTGTTCAATAACCCAAACAT
CAAAGGGACTTGTGGCTGTGGAGAAAGCTTTAATATTTGA

It is known by a skilled artisan that the codon systems in different organisms can be slightly different, and that therefore where the expression of a given protein from a given organism is desired, the nucleic acid sequence can be modified for expression within that organism.

In a preferred embodiment of the invention, the MAR coding sequence from pigeon was optimized in terms of codon usage for expression in C. elegans, and two artificial introns were added so as to enhance its expression (see, e.g., SEQ ID NO:11).

An aspect of the present invention provides nucleic acid sequences that code for pigeon MAR protein that is optimized for expression in, e.g., mouse, rat, marmoset and human (see, e.g., SEQ ID NOs:7-10).

SEQ ID NO: 7, synthetic codon-optimized pigeon MAR
gene for expression in mouse.
ATGGCCTCTAGCGCCTCTAGCGTCGTGAGAGCTACCGTGAGAGCCGTGTC
CAAGAGGAAGATCCAGGCCACAAGAGCCGCTCTGACACTGACCCCTAGCG
CCGTGCAGAAGATCAAGGAGCTGCTGAAGGACAAGCCCGAACACGTGGGA
GTGAAAGTGGGCGTGCGAACCAGAGGTTGCAACGGCCTGAGCTACACCCT
GGAGTACACCAAGAGCAAGGGCGACAGCGACGAGGAAGTGGTGCAGGACG
GAGTGCGAGTGTTCATCGAGAAGAAGGCCCAGCTGACACTGCTGGGAACC
GAGATGGACTACGTGGAGGACAAGCTGAGCAGCGAGTTCGTGTTCAACAA
CCCCAACATCAAGGGCACTTGCGGCTGCGGCGAGTCTTTTAACATCTAG
SEQ ID NO: 8, synthetic codon-optimized pigeon MAR
gene for expression in rat.
ATGGCCTCTAGCGCCTCTAGCGTCGTGAGAGCTACCGTGAGAGCCGTGTC
CAAGAGGAAGATCCAGGCCACAAGAGCCGCTCTGACACTGACCCCTAGCG
CCGTGCAGAAGATCAAGGAGCTGCTGAAGGACAAGCCCGAACACGTGGGA
GTGAAAGTGGGCGTGCGAACCAGAGGTTGCAACGGCCTGAGCTACACCCT
GGAGTACACCAAGAGCAAGGGCGACAGCGACGAGGAAGTGGTGCAGGACG
GAGTGCGAGTGTTCATCGAGAAGAAGGCCCAGCTGACACTGCTGGGAACC
GAGATGGACTACGTGGAGGACAAGCTGAGCAGCGAGTTCGTGTTCAACAA
CCCCAACATCAAGGGCACCTGCGGATGCGGCGAGTCTTTTAACATCTAG
SEQ ID NO: 9, synthetic codon-optimized pigeon MAR
gene for expression in marmoset.
ATGGCTAGCAGCGCTAGCAGCGTGGTGAGGGCTACCGTGAGGGCCGTGTC
CAAGAGGAAGATCCAGGCTACCAGGGCCGCTCTGACTCTGACTCCAAGCG
CCGTGCAGAAGATCAAGGAGCTGCTGAAGGACAAGCCCGAACACGTGGGA
GTGAAGGTGGGAGTGAGGACCAGGGGTTGTAACGGCCTGAGCTACACCCT
GGAGTACACCAAGAGCAAGGGCGACAGCGACGAGGAAGTGGTGCAGGACG
GAGTGAGGGTGTTCATCGAGAAGAAGGCTCAGCTGACCCTGCTGGGAACC
GAGATGGACTACGTGGAGGACAAGCTGAGCAGCGAGTTCGTGTTCAACAA
CCCCAACATCAAGGGCACTTGCGGTTGCGGCGAGAGCTTTAACATCTAA
SEQ ID NO: 10, synthetic codon-optimized pigeon MAR
gene for expression in human.
ATGGCTAGCAGCGCCTCTAGCGTCGTGAGAGCTACAGTGCGGGCCGTGTC
CAAGAGAAAGATCCAGGCCACAAGAGCCGCTCTGACACTGACCCCTAGCG
CCGTGCAGAAGATCAAGGAGCTGCTGAAGGACAAGCCCGAACACGTGGGA
GTGAAAGTGGGCGTGAGGACCAGAGGTTGCAACGGCCTGAGCTACACCCT
GGAGTACACCAAGAGCAAGGGCGACAGCGACGAGGAAGTGGTGCAGGACG
GAGTGCGAGTGTTCATCGAGAAGAAGGCCCAGCTGACACTGCTGGGAACC
GAGATGGACTACGTGGAGGACAAGCTGAGCAGCGAGTTCGTGTTCAACAA
CCCCAACATCAAGGGCACTTGCGGCTGCGGCGAGTCTTTTAACATCTAG

Another aspect of the present invention provides reagents for genetically targeted expression of the MAR protein. Genetic targeting can be used to deliver MAR gene to specific cell types, to specific spatial regions within an organism, and to sub-cellular regions within a cell. Genetic targeting also relates to the control of the amount of MAR protein expressed, and the timing of the expression.

A preferred embodiment of a reagent for genetically targeted expression of the MAR protein comprises a vector which contains the gene for the MAR protein.

As used herein, the term “vector” refers to a nucleic acid molecule capable of transporting between different genetic environments another nucleic acid to which it has been operatively linked. The term “vector” also refers to a plasmid, a virus or organism that is capable of transporting the nucleic acid molecule. One type of preferred vector is an episome, i.e., a nucleic acid molecule capable of extrachromosomal replication. Preferred vectors are those capable of autonomous replication and/or expression of nucleic acids to which they are linked. Vectors capable of directing the expression of genes to which they are operatively linked are referred to herein as “expression vectors”. Other preferred vectors are viruses such as lentiviruses, retroviruses, adenoviruses, adeno-associated viruses, rabies viruses, herpes simplex viruses and phages. Preferred vectors can genetically insert MAR gene in-vivo or in-vitro.

As used herein, the term “subject” refers to an animal, preferably a mammal, such as a human, but can also be other animals, e.g., zebrafish, flies, worms, mice, rat, and marmoset.

Expression vectors compatible with eukaryotic cells can also be used. Eukaryotic cell expression vectors are well known in the art and are commercially available. Typically, such vectors are provided containing convenient restriction sites for insertion of the desired DNA homologue.

One preferred expression vector of the present invention comprises the MAR gene and mec-4 promoter.

One aspect of the invention is a nucleic acid sequence comprising the gene for MAR protein and a promoter for genetically targeted expression of the MAR protein. The genetically targeted expression of the MAR protein can be facilitated by the selection of promoters. The term “promoter” as used herein is nucleic acid sequence that enables a specific gene to be transcribed. The promoter usually resides near a region of DNA to be transcribed. By use of the appropriate promoter, the level of expression of MAR protein can be controlled. Cells use promoters to control where, when, and how much of a specific protein is expressed. Therefore, by selecting a promoter that is selectively expressed predominantly within one type of cell, one subtype of cells, a given spatial region within an organism, or sub-cellular region within a cell, the control of expression of MAR can be controlled accordingly. The use of promoters also allows the control of the amount of MAR expressed, and the timing of the expression. The promoters can be prokaryotic or eukaryotic promoters.

One embodiment of the present invention is a nucleic acid sequence comprising the gene for MAR protein and a cell specific promoter. Examples of cell specific promoters are promoters for somatostatin, parvalbumin, GABAα6, L7, and calbindin. Other cell specific promoters are promoters for kinases such as PKC, PKA, and CaMKII; promoters for other ligand receptors such as NMDAR1, NMDAR2B, GluR2; promoters for ion channels including calcium channels, potassium channels, chloride channels, and sodium channels; and promoters for other markers that label classical mature and dividing cell types, such as calretinin, nestin, and beta3-tubulin.

Cells

The cells of the present invention can be created using a vector including a DNA expression vector, a virus or an organism. Preferred vectors include plasmids, lentiviruses and retroviruses. In some cases, in particular where robust cell lines are involved, expression of MAR can be induced by using lipofection techniques, such as exposing cell lines to micelles containing Lipofectamine or Fugene, and then FACS-sorting to isolate stably expressing cell lines.

Cells of any origin, preferably those cells that are capable of growth in tissue culture, are candidate cells for transfection or infection with a MAR gene. Non-limiting examples of specific cell types that can be grown in culture include fibroblast, skeletal tissue (bone and cartilage), skeletal, cardiac and smooth muscle, epithelial tissues (e.g. liver, lung, breast, skin, bladder and kidney), neural cells (glia and neurones), endocrine cells (adrenal, pituitary, pancreatic islet cells), bone marrow cells, and melanocytes. Suitable cells can also be cells representative of a specific body tissue from a subject. The types of body tissues include, but are not limited, to blood, muscle, nerve, brain, heart, lung, liver, pancreas, spleen, thymus, esophagus, stomach, intestine, kidney, testis, ovary, hair, skin, bone, breast, uterus, bladder, spinal cord and various kinds of body fluids. Cells of different developmental stages (embryonic or adult) of an organism, or more specifically of various developmental origins including ectoderm, endoderm and mesoderm; can also be applied.

Of particular interest are cells that are associated with a particular disease or with a specific disease stage, cells derived from natural and induced immune deficiency states, cardiovascular disease, neuronal disease, inflammation states and diseases caused by a variety of pathogens. A disease cell may also be confirmed by the presence of a pathogen causing the disease of concern (e.g. HIV for AIDS and HBV for hepatitis B).

Preferred cells are mammalian cells and cell lines derived from mammalian cells. Other preferred cells are embryonic stem cells and adult stem cells including hematopoetic stem cells, bone marrow, neural stem cells, epithelial stem cells, skin stem cells. Preferred cell lines appropriate for MAR expression include, HEK cells, neural stem cell lines, pancreatic islet cell lines, and other excitable or secretory cells.

Cell viability may be confirmed by the measurement of membrane integrity. The methods for assessing membrane integrity are known in the art.

Transgenic Animals

One aspect of the invention is a transgenic animal that expresses MAR protein. Expression of MAR protein in particular subsets of neurons can be used for analyzing circuit function, behavior, plasticity, and animal models of psychiatric disease.

Preferred transgenic animal species of the present invention that expresses MAR protein include zebrafish (Danio rerio), flies (e.g. Drosophila melanogaster), worms (e.g. Caenorhabditis elegans), mice, rat, and marmoset.

Another preferred transgenic animal species of the present invention that expresses MAR protein is mice. In one preferred embodiment, mice that express MAR protein are made using BAC (bacterial artificial chromosome) transgenic technology, as well as position effect variegation techniques.

One preferred embodiment of a transgenic animal of the present invention that expresses MAR protein is Caenorhabditis elegans.

Another preferred embodiment of the present invention is a transgenic animal wherein the MAR is expressed under a specific promoter. Another preferred embodiment of the present invention is a transgenic animal wherein the MAR expressed in the transgenic animal is introduced via a BAC. Another preferred embodiment of the present invention is a transgenic animal wherein the MAR gene is knocked into a known locus.

Methods of Treatment

Another aspect of the invention is a method for treating a subject comprising delivering a vector comprising a MAR gene to, e.g., excitable cells within the subject and exposing said cells to an external magnetic field.

A preferred embodiment of a method for treating a subject comprises performing human therapeutic functions in which the function of cells is rescued or controlled by the genetic addition of MAR, accompanied by the use of physically delivered magnetic stimulation. Delivering a MAR protein in human patients via viral vectors can enable control of excitable cells by magnetic stimulation. For example, peripheral neurons like cutaneous pain suppressing nerves, virally transduced to express MAR, allow magnetic stimulation to activate dorsal column-medial lemniscus neurons in order to suppress painful C fiber responses. Modified herpes viruses can be used to deliver MAR to pain-pathway neurons. Similarly, patients who have rod or cone loss (such as in retinitis pigmentosa or macular degeneration) can be virally transduced to express a MAR protein in retinal ganglion cells, which restores the transduction of light in pathways mediating visual perception. Thus, the strategy based on the expression of MAR is suitable for retinal degenerative diseases.

In one embodiment, the magnetic devices used to excite MAR protein-expressing cells in patients are commercially available. Any conventional magnetic devices which produce a magnetic field can be used in the present invention to stimulate the MAR protein-expressing cells.

The methods and compositions provided herein can provide a beneficial effect for Alzheimer's patients. Preferably Alzheimer's patients are treated by delivering and exciting MAR protein to brain of human patients by the methods described herein.

Similarly, the methods and compositions provided herein can provide a beneficial effect for Parkinson's patients. Preferably Parkinson's patients are treated by delivering and exciting a MAR protein to the subthalamic nuclei and/or globuspallidus of human patients by the methods described herein.

Another route for human therapy using a MAR protein is to create a MAR protein-expressing secretory cell for implantation in patients (for example, nanoencapsulated to avoid immune responses) in which secretion is stimulated in the cells by the use of physically delivered magnetic stimulation. For example, MAR-expressing neuroendocrine cells that release thyroid hormones (such as T4, TRH, and others) can be implanted subcutaneously to allow for controlled peptide release over timescales from months to years. Similarly, MAR protein-expressing pancreatic islet cells can be made to release insulin when stimulated with a remote magnetic field; implanted cells can enable control of diabetes symptoms on a minute-to-minute timescale without need for pump implantation or other invasive therapy.

In one embodiment, MAR protein-expressing cells are encapsulated prior to implantation into patients. The cells can be macroencapsulated or nanoencapsulated. Examples of capsules include but are not limited to semipermeable membranes, hollow fibers, beads and planar diffusion devices.

In another embodiment, differentiated MAR protein-expressing stem cells capable of secreting dopamine would be implanted, directly into the brain of a patient, and then drive their activation using magnetic stimulation. Dopamine-secreting cells can be transfected or infected as described herein with MAR protein, before or after the differentiation step, and then these cells can be implanted into the brain of the patients.

In another embodiment, MAR protein-expressing secretory cells are implanted into a tissue or an organ of a patient. The secretory cell is transfected or infected as described herein with MAR, and then these cells are implanted into the tissue or organ of the patient. The MAR protein-expressing secretory cells are then induced to secrete chemicals by a magnetic device.

Examples of tissues or organs that can be implanted with MAR protein-expressing secretory cells include, but are not limited to epithelium, connective tissue, nervous tissue, heart, lungs, brain, eye, stomach, spleen, pancreas, kidneys, liver, intestines, skin, uterus, and bladder.

In one embodiment, MAR protein-expressing secretory cells are implanted into the skin of a diabetic or patient. The MAR protein-expressing secretory cells are then induced to secrete insulin by a magnetic device.

### Examples

Introduction

Although the mechanism for magnetoreception has remained elusive, it has been proposed that to maximize the ability of animals to sense the geomagnetic fields, cryptochrome (Cry), a well-known magnetosensor needs to interact with another magnetoreceptor. There are many candidate interacting partners for Cry to form a stable complex, and one of which is Isca1, a iron-sulfur cluster assembly 1 (Isca1) protein (Cózar-Castellano et al., 2004). Since Isca1 gene is very conserved among C. elegans, fruit fly, butterfly, zebrafish, pigeon, chicken, rat, mouse, dog, cow, chimpanzee and human with very high homology, it might bind to Cry and play an important role in electron-transfer reactions. We hypothesize that Isca1-carried electron-transfer reactions might trigger action potentials in Isca1-expressing cells and induce neuronal activity after stimulation with the external magnetic field. We re-define these kinds of highly conserved iron-sulfur assembly proteins as magnetoreceptor (MAR), which can be magnet-responsive. The MAR family includes all of highly conserved Isca1 homologues across different species. In this study, we invented a non-invasive technique named as magnetogenetics thereafter, which combines the genetic targeting of a magnetoreceptor with remote magnetic stimulation. Since pigeon has the strongest magnetic sensing system, we therefore express the pigeon Isca1 and four different codon-optimized versions for mouse, rat, marmoset and human (see SEQ ID Nos:7-10) to explore our magnetogenetics in vivo and in vitro. We found that Isca1 could evoke membrane depolarization and action potentials, generate calcium influx and trigger neuronal activity in both HEK-293 and cultured primary hippocampal neurons when activated by a remote magnetic field. The successful combination of remote magnetic stimulation and genetic targeting will, therefore, reshape the landscape of currently available neuroperturbation and neuromodulation toolboxes including optogenetics and deep brain stimulation. This novel technology makes the exclusively non-invasive dissection of complex brain circuitry as well as the modulation of deep brain regions possible, opening a new door to non-invasive, remote and magnetic control of neuronal activities in the intact mammalian brains and biological processes in other organisms.

### Experimental Procedures

DNA Constructs

All plasmids were constructed by standard molecular biology procedures and subsequently verified by double strand DNA sequencing. GCaMP6s and ASAp1 were from Addgene. The AAV-CAG-MAR-P2A-GCaMP6s and Lenti-CAG-MAR-P2A-GCaMP6s were connected via a 2A peptide (P2A) under the chimeric promoter CAG (a combination of the cytomegalovirus early enhancer element and chicken beta-actin promoter). ASAP1 expression plasmid (pcDNA3.1/Puro-CAG-ASAP1) was from Addgene 52519. The AAV-CAG-MAR-P2A-ASAP1 and Lenti-CAG-MAR-P2A-ASAP1 were created with multiple PCR cloning.

HEK-293 and Transfection

HEK-293 cells were maintained and continuously passaged with high-glucose Dulbecco's Modified Eagle Medium (DMEM, Gibco/BRL) containing fetal bovine serum (FBS, Life Tech). Transfection was performed using either Lipofectamine-2000 (Life Tech) or classical calcium phosphate transfection.

Primary Neuronal Culture and Transfection

Rat hippocampus were dissected from embryonic day 18 rats, and primary cultured hippocampal neurons were cultured has been described (Zhang et al., 2007; Du et al., 2000). Transfection was performed using either Lipofectamine-2000 (Life Tech) or classical calcium phosphate transfection at different days of in vitro culture.

rAAV Production

The rAAV vector was pseudotyped with AAV1 capsid (Zhang et al., 2011). The chimeric rAAV2/1 was prepared by co-transfection of human embryonic kidney cell line HEK-293 prepared from co-transfection using the standard calcium phosphate method along with the adenoviral helper plasmid pHelper (Strategene, CA, USA). Twelve hours after transfection, the DNA/CaCl₂ mixture was replaced with normal growth medium. After an additional 60 hours in culture, the transfected cells were collected and subjected to three times of freeze/thaw. The clear supernatant was then purified using heparin affinity columns (HiTrap Heparin HP, GE Healthcare, and Sweden). The purified rAAV2/1 was concentrated with an Amicon Ultra-4 centrifugal filter 100K device (Millipore, MA, USA), and the viral titer was determined by real-time quantitative PCR using StepOnePlus Real-Time PCR Systems and TaqMan Universal Master Mix (Applied Biosystems, CA, USA). The titered virus was diluted and titer-matched to 1.0×10¹² viral genomic particles/ml by 1×phosphate-buffered saline.

Immunofluorescent

For the immunostaining, sections were rinsed three times for 10 min in 1×PBS at room temperature and pre-incubated for 2 hours in 10% normal goat serum in PBST (1×PBS with 0.5% Triton X-100). All rinses between incubation steps were with PBST (Zhang et al., 2009). After rinsing, processed sections were incubated with different primary antibodies against MAR (Home-made, 1:200), NeuN (Millipore, 1:500) and mCherry (Clontech, 1:2000) for 72 hours in antibody-blocking buffer at 4° C. After three times of 15-min washing in 1×PBST at room temperature, sections were incubated in a secondary antibody conjugated with either fluorescein isothiocyanate or Cy3, respectively (Jackson ImmunoResearch, West Grove, Pa., USA, 1:2000) for 2 hours at room temperature. After intensive rinse with 1×PBST, sections were mounted onto glass slides, and a cover slip was applied.

Growth and Transgenesis of C. elegans Lines

All C. elegans strains were grown and maintained on nematode growth media (NGM) agar plates cultured at 20° C. The NGM agar plates were seeded with OP50 Escherichia coli. Transgenic strains were generated through a standard micro-injection into N2 worms according to a standard procedure (Evans et al., 2006). Untagged MAR in transgene zdEx12[pmyo-3::MAR; pmyo-3::gfp] and zdEx22[pmec-4::MAR; pmec-4::gfp; sur-5::mCherry] were injected in N2, yielding strains that carried extrachromosomal arrays ZD24, ZD34, respectively. The plasmids pmyo-3::gfp, pmec-4::gfp and sur-5::mCherry were co-injected as markers to make sure those specific cells were successfully inherited with the transgenic array. The certain promoter driven GFP (two strains for myo-3 and mec-4, see Table S1) was used to monitor the expression pattern of MAR. The behavior of C. elegans in response to the magnetic stimulation was recorded under bright field illumination.

TABLE S1
C. elegans transgenes and strains.

| Transgene | Genotype | Strain |
|---|---|---|
| zdEx12[pmyo-3:: MAR; pmyo-3::gfp] | N2 | ZD24 |
| zdEx22[pmec-4:: MAR; pmec-4::gfp; sur-5::mCherry] | N2 | ZD34 |

Whole-Cell Clamp Recording in Cultured Hippocampal Neurons

Neurons were recorded with Axon MultiClamp 700B amplifier (Axon Instruments, USA) immersed in Tyrode's solution (Boyden et al., 2005). The intracellular solution of glass pipettes (resistance in the range of 3-8 MO) contained (in mM): 125 potassium gluconate, 0.5 EGTA, 4 magnesium ATP, 5 NaCl, 0.3 sodium GTP, 10 phosphocreatine, 10 HEPES (pH 7.2 with KOH). In FIG. 5C where voltage-clamp were made, intracellular solution consisted of (in mM) 125 Cs-gluconate, 4 magnesium ATP, 0.3 sodium GTP, 10 phosphocreatine, 10 HEPES, 0.5 EGTA, 3.5 QX-314, 5 TEA, 2 CsCl (pH 7.2 with NaOH). Inward and outward currents were recorded while clamping neurons at −70 mV and 0 mV, respectively. Membrane resistance was measured by injecting a 10 mV step lasting 100 ms in voltage-clamp mode.

Calcium Imaging

Calcium imaging was performed with Olympus BX61WI upright microscopy equipped with a 40× water-immersion objective and an Olympus DP-80 CCD. The relative change of fluorescence intensity (ΔF/F0) was extracted using ImageJ. Heat map was generated using Matlab (MathWorks, USA).

Results

Induction of Calcium Influx by MAR Via a Magnetic Field in HEK-293

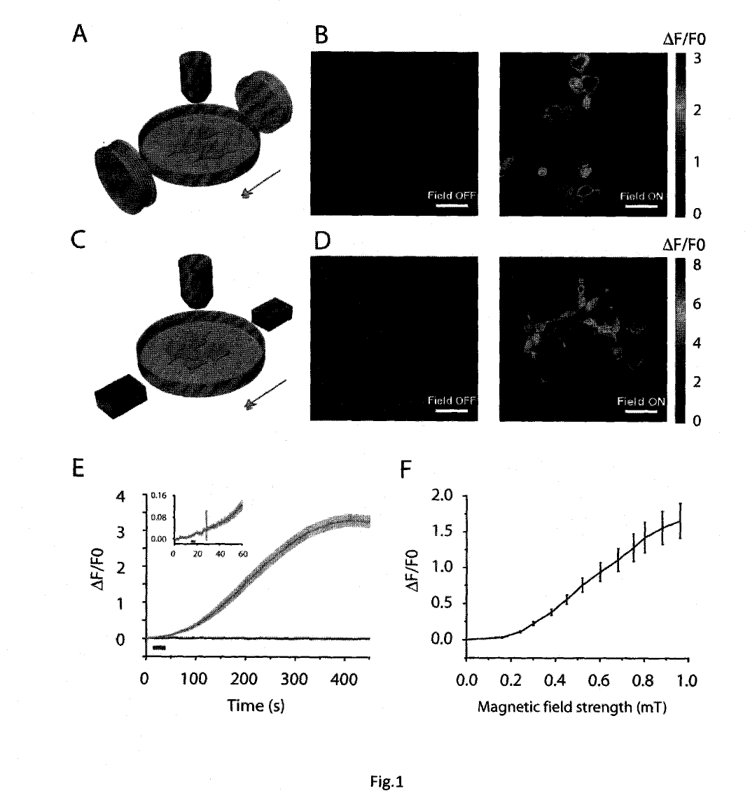

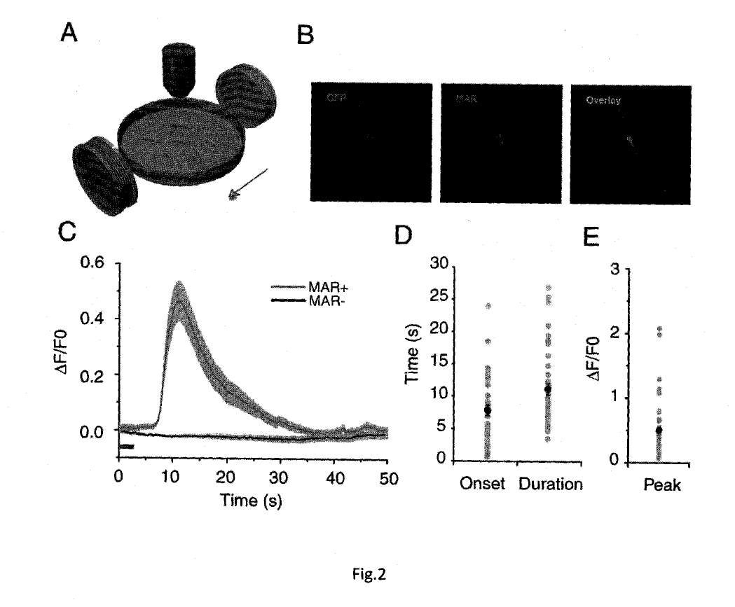

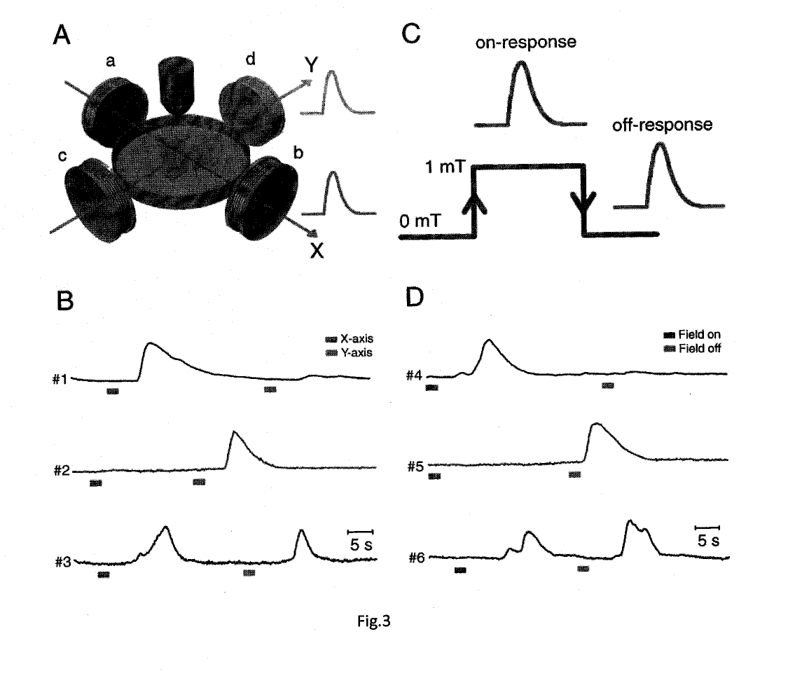

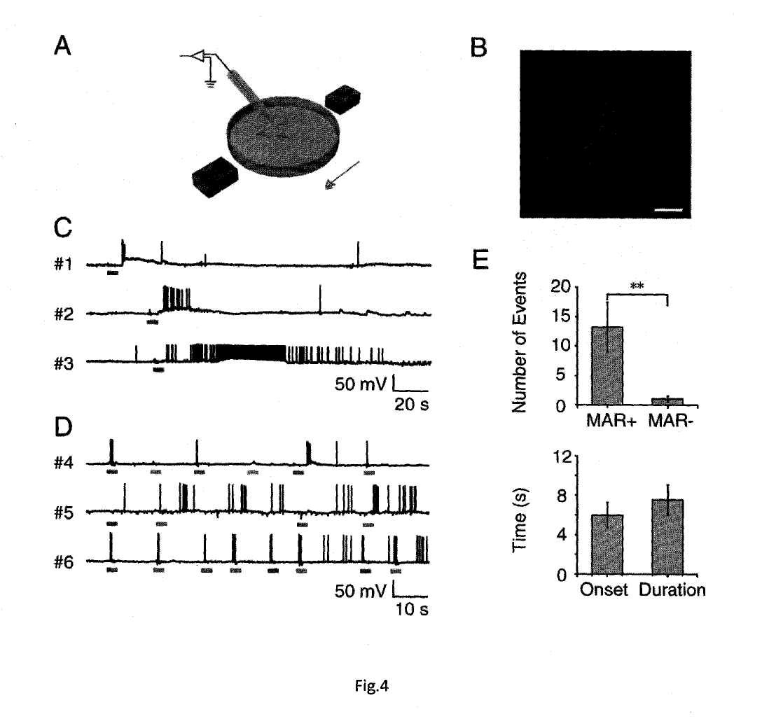

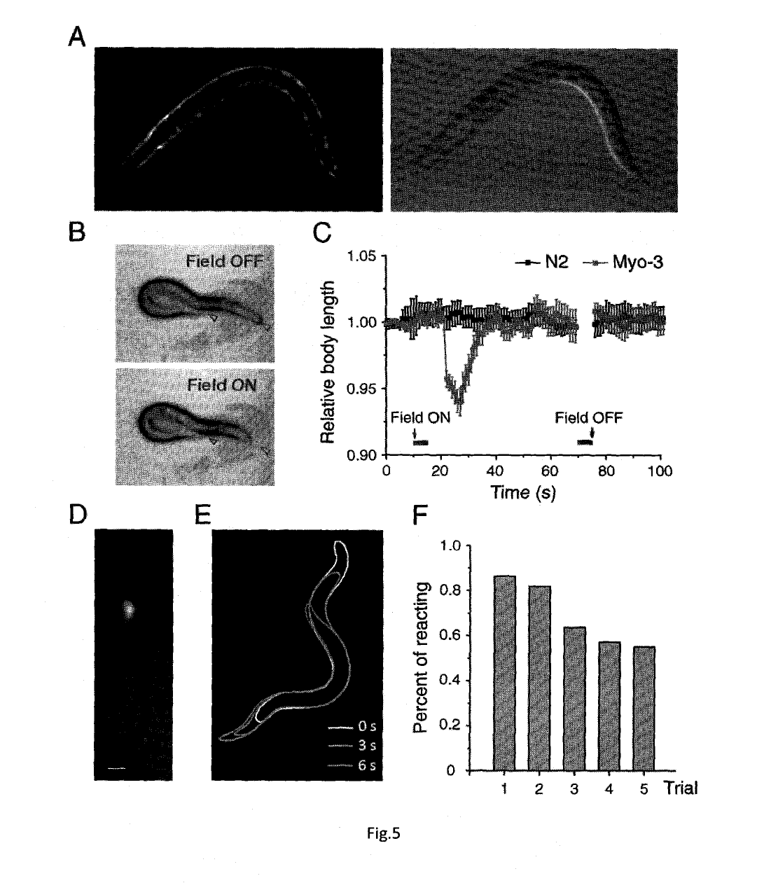

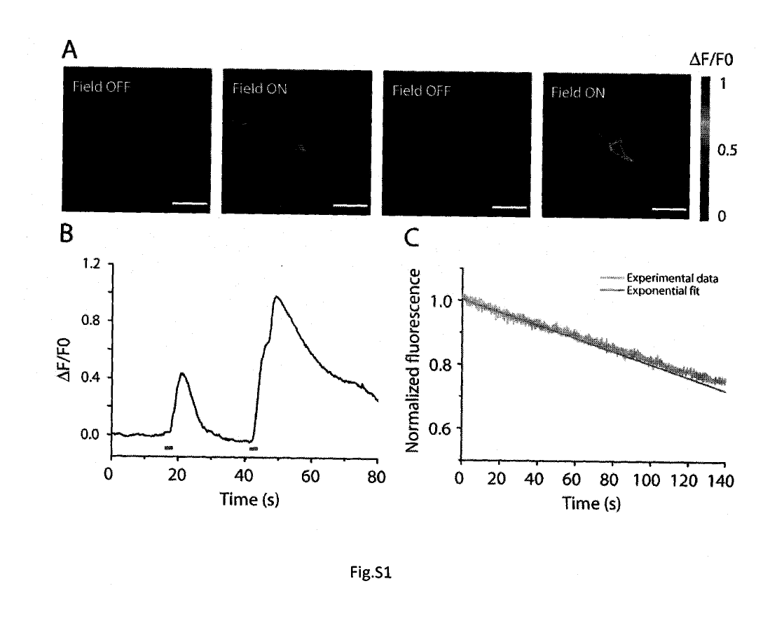

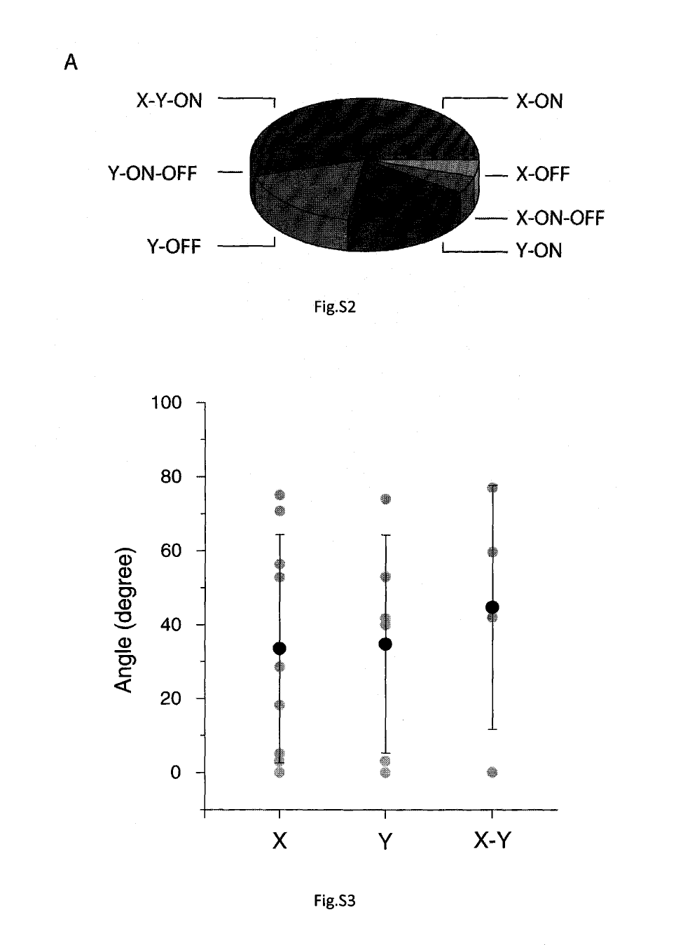

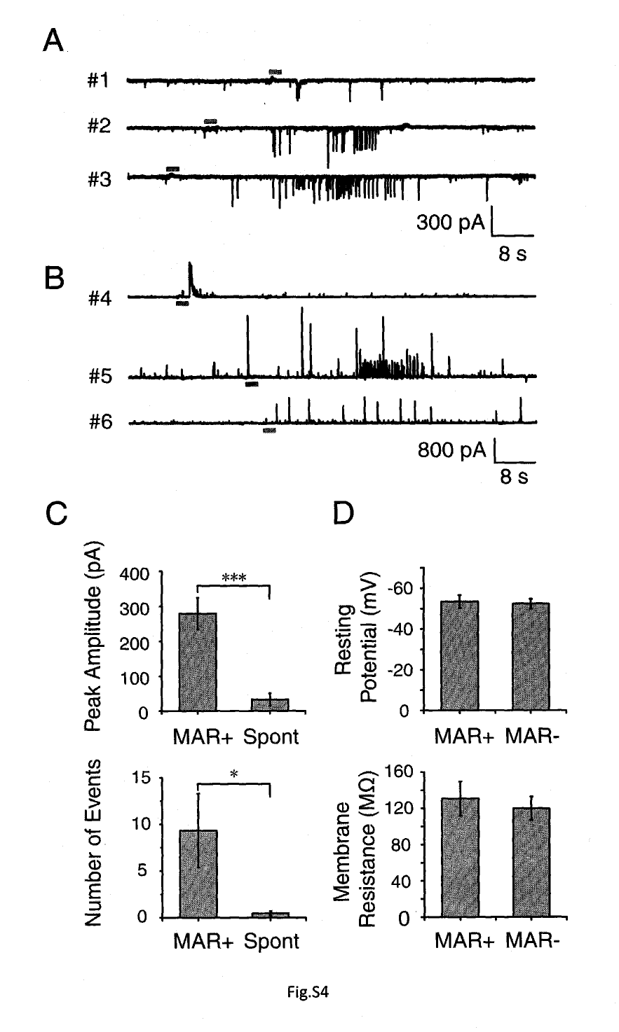

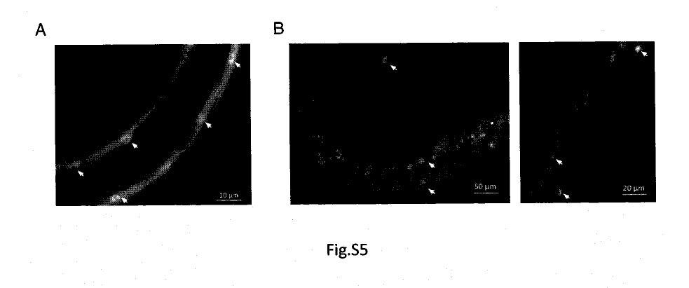

We explored whether MAR could function as a magnet-responsive protein and therefore can be used for the magnetogenetic control of neuronal activity with a remote magnetic field. We first co-transfected this MAR with the genetically encoded and ultrasensitive calcium indicator GCaMP6s (Chen et al., 2013; Tian et al., 2009) into the human embryonic kidney (HEK)-derived cell line HEK-293 cells. We constructed a custom-made magnetic generator consisting of two pairs of coils, which can hold a standard 35-mm culture dish (FIG. 1A). Our home-made magnetic generator can produce a maximum magnetic field strength of about 1 millitesla (mT) at the center of the dish and approximately 2.5 mT on the edge. Cells at different positions in the culture dish receive different amount of magnetic field strength when stimulated with either our home-made magnetic device or hand-held static magnetic bars (FIG. 1C).

Before we turned on the magnetic generator, the fluorescence intensity of GCaMP6s in HEK-293 cells remains stable at a base level. After applying the magnetic field, we detected a dramatic increase in fluorescence intensity in MAR-transfected HEK-293 cells (FIG. 1B), showing almost 350% increase compared with the base fluorescence intensity (FIG. 1E). The fluorescence intensity increased to over 10 times of the standard deviation of the base fluorescence intensity, with an average duration of 13 s, indicated by the gray dashed line in the inset of FIG. 1E. Importantly, no increase was observed in control group without the expression of MAR (FIG. 1E).

We measured the threshold of magnetic strength by testing the changes of fluorescence intensity in response to magnetic field strength ranging from 0 to 1 mT measured at the center of the culture dish from our home-made device (FIG. 1F). To activate MAR-transfected HEK-293 cells, the minimum magnetic strength required was near 0.3 mT which was about 6 times higher than the earth's magnetic strength (˜50 μT) (Mouritsen and Ritz, 2005). No increase was observed when only the earth's magnetic field under our working environment was present (data not shown), indicating that the geomagnetic field could not activate MAR and a relative strong magnetic field was needed to elicit response in MAR-transfected cells. Compared to the strong magnetic field strength of up to several Tesla in diagnostic and therapeutic fMRI (Logothetis, 2008), the magnetic strength present in our study to stimulate MAR was at a level of only several millitesla, suggesting that MAR-dependent magnetogenetic control is not only robust against the influence from geomagnetic field but also safe.

To eliminate the possible artifact due to the background interference from potential fluctuations in the magnetic field generated by the electrical coils of our home-made magnetic generator, we replaced our home-made magnetic generator with hand-held static magnetic bars (FIG. 1C) producing almost 2.5 mT at the center of the dish, and found the same observation of dramatic fluorescence increase as that induced by the magnetic generator (FIG. 1D). These observations together suggest that the magnetoreceptor (MAR) functions as a magnet-responsive activator, depolarizes membrane potentials and subsequently generates calcium influx in a magnetic field-dependent way.

MAR-Evoked Calcium Influx in Neuron

We next asked whether MAR can activate neurons and induce calcium influx in MAR-transfected neurons after the application of the external magnetic fields. We co-transfected or infected the primary cultured rat hippocampal neurons using MAR together with GCaMP6s (Du et al., 2010; Zhang et al., 2007) when enriched processes were formed functionally. The immunofluorescent staining showed that MAR appeared to be expressed mainly somato-dendritically (FIG. 2B). The MAR-negative neurons showed almost no detectable MAR expression, indicating MAR was produced exogenously not endogenously at least in the hippocampal neurons. Similarly, we could observe the potentiation of Ca²⁺ transients (ΔF/F0=50.5±7.0%, n=42, FIG. 2E) within 7.8±0.8 s (FIG. 2D) after the onset of the externally applied magnetic field (FIG. 2A). Traces were corrected for photobleaching described in FIG. S1C. The duration of GCaMP6s in MAR-transfected cultured neurons lasted 11.1±0.9 s (FIG. 2D). As a control, no significant increase in calcium spiking was observed in MAR-negative neurons (n=48, FIG. 2C). We found the minimum magnetic strength required to activate the neurons was similar to that in HEK-293. Furthermore, we could repeatedly activate both MAR-transfected and infected neurons and detected similar patterns of calcium spike train (Figures S1A and S1B), suggesting that the magnetic activation of neuronal activity is also quickly reversible. Thus, the magnetogenetic activation of MAR could depolarize neuronal membrane and trigger action potentials quickly and reversibly.

Magnetic Direction-Selective Control of Neuronal Activity

Since magnetic field has orientation (Winkhofer et al., 2012), we reasoned that magnetogenetic control of evoked action potentials might be affected by the direction of the external magnetic field applied. To investigate this possibility, we tested the neuronal responses to magnetic fields with different directions. We first checked whether the direction of the applied magnetic field affected the MAR-evoked response of calcium transients of GCaMP6s in our two-dimensional coil-based magnetic generator (FIG. 3A). Since the magnetic field was produced by only one of two pairs of orthogonal coils (a-b and c-d) each time in our home-made magnetic device, we generated magnetic fields along either one of the orthogonal directions, that is, the X-direction (from a to b) and the Y-direction (from c to d).

We observed that 7 out of those 22 magnet-responsive neurons were activated only by magnetic field along the X-direction (FIG. 3B, upper panel), while 11 out of those 22 neurons were activated only by magnetic field along the Y-direction (FIG. 3B, middle panel). Interestingly, the four remaining neurons (4/22) displayed robust calcium spikes in response to both magnetic fields along the X-direction and along the Y-direction (FIG. 3B, lower panel). We further quantified whether the correlation between the axonal orientation of MAR-transfected neurons and the direction of the applied magnetic field influenced the MAR-triggered responses. No obvious correlation was found between the MAR-triggered response and the axonal orientation relative to the direction of the applied magnetic field (FIG. S3). Since we also found the similar magnetic direction-dependent effect in HEK-293 cells, such directional effect might not be neuron-specific rather rod-like rearrangement of expressed MAR on the cellular membrane due to magnetic stimulation. We could not exclude the possibility that expression level of MAR, rod-like cluster redistribution of magnet-induced MAR on the cellular membrane, higher magnetic strength and/or uniform magnetic activation might eliminate such magnetic direction-dependent heterogeneous effect on neuronal activation. These observations suggested that the magnetogenetic control of action potentials might depend on the direction of the external magnetic field applied in our particular setup given that the maximal magnetic strength cannot exceed 1 mT in our own home-made device. It would be interesting to test the effect of magnetic polarity on neuronal activity with more sophisticated magnetic device in our future experiments.

On-Response and Off-Response Effect of Magnetic Field on Neuronal Activity

Since turning the magnetic field on or off might change membrane extension and then open some ion channels in the membrane, we hypothesized that the onset or the offset of the external magnetic field applied could also affect neuronal activity (Winkhofer et al., 2012). As expected, we found the on-response, off-response and on/off-response patterns of neuronal activity when magnetic field is switched on or off (FIG. 3C) in those 22 neurons tested above. We found 12 out of those 22 MAR-GCaMP6s-co-transfected neurons showed dramatic increase in fluorescence intensity when the magnetic field was switched on only. However, the increased calcium transients went back to the base level (FIG. 3D, upper panel) when the magnetic field was turned off. Interestingly, to the opposite, 6 out of those 22 MAR-transfected neurons showed no increased activity after the onset of the magnetic field while GCaMP6s fluorescence showed transient increase when the magnetic field was switched off for the same group of neurons (FIG. 3D, middle panel). Interestingly, a small group of neurons (n=4) responded as actively when the magnetic field was switched from on to off as from off to on (FIG. 3D, lower panel). The distribution of the four different response patterns was summarized in FIG. S2. We could not exclude the possibility that heterogeneous expression of MAR within neurons or rod-like iron-sulfur cluster rearrangement from magnet-stimulated MAR on the cellular membrane and/or non-uniform distribution of magnetic field in our home-made magnetic generator might cause such differential on-off responses of neuronal activity (Winkhofer et al., 2012). Future experiments should be performed with a magnetic generator with higher power and more precise control.

MAR Elicits Magnetocurrent and Spiking in Neuron

We further examined whether magnet-stimulated MAR can depolarize neurons and evoke a train of action potentials in cultured hippocampal neurons using both voltage-clamp and current-clamp (FIG. 4A) with a pair of hand-held static magnetic bars (Mora et al., 2004; Semm and Beason, 1990), which was used to avoid interference from potential fluctuations in the magnetic field generated by the electrical coils of our home-made device. We transfected neurons with a P2A-linked MAR-mCherry driven by a chicken beta actin-CMV chimeric promoter (CBA), ensuring all identified, mCherry-positive neurons are co-expressed with MAR (FIG. 4B).

Magnetic field evoked rapid inward currents in MAR-positive neurons. Representative recordings showed that whole-cell currents were elicited by application of magnetic field in mCherry-positive neurons clamped at −70 mV (FIG. S4A, traces#1-3). Mean inward peak current was 279.6±45.2 pA and the average number of events was 9.3±3.95 (FIG. S4C). Since magnetic field tended to stimulate both excitatory and inhibitory neurons expressing MAR in the culture dish, outward currents could also be recorded in neurons that were voltage clamped at 0 mV (Jackson, 2001) (FIG. S4B, traces#4-6).

We next investigated whether MAR could drive neuronal firing in a current-clamp mode with the same stimulus used for eliciting magneto-current above. Voltage traces shown in FIG. 4C were three representative neurons (traces#1-3) with the increase of firing rate stimulated by external magnetic field. Consistent with those results (FIG. 3D) obtained from calcium imaging, we also observed three similar On-Off firing patterns stimulated with external magnetic field (FIG. 4D): one activated with on-response only, the second one with off-response only and the third one with both on-response and off-response. Population data showed that the number of spikes evoked by MAR was significantly higher than spontaneous events (n=19; **, P=0.003, student t-test), with 13.2±4.2 spikes versus 1.0±0.5 spikes. The spike trains lasted for 8.5±1.5 s with 5.3±1.1 s delay after field onset (FIG. 4E). We quantified the intrinsic electrical properties by injecting a 10 mV voltage step under voltage-clamp mode in MAR-positive and MAR-negative neurons. Both resting membrane potential and membrane resistance showed no significant difference between neurons expressing MAR and those not expressing MAR (FIG. S3D). Thus, MAR was able to induce membrane depolarization quickly, evoke action potentials repeatedly and control neuronal activity remotely.

MAR can Trigger Locomotion and Induce Withdrawal Behaviors in C. elegans

To test whether the magnet-dependent activation of MAR can trigger circuit and network behaviors in transgenic animals, we constructed transgenic nematode Caenorhabditis elegans by expressing MAR under the control of the promoter myo-3, which restricts its expression to the muscle cells in C. elegans (Nagel et al., 2005). To improve the expression level of MAR in C. elegans, we synthesized an artificial MAR gene by optimizing its codon usage, based on its deduced amino acid sequence from pigeon, and by adding two artificial introns that was confirmed to enhance its expression in C. elegans (Husson et al. 2013; Liu et al., 2009; Okkema et al., 1993)(SEQ ID NO:11). MAR expression was restricted to muscle cells under the promoter of myo-3 (FIG. 5A and FIG. S5A).

SEQ ID NO: 11, synthetic MAR gene used in
C. elegans. The two regions in bold and lower-case
letters represent the two artificial introns.
The regions in capital letters represent the codon-
optimized coding sequence of MAR for expression in
C. elegans.
ATGGCTTCGTCGGCCTCATCAGTTGTTAGAGCTACAGTTCGAGCCGTGTC
GAAGCGAAAGATTCAAGCTACACGAGCTGCCCTCACACTCACACCATCAG
CTGTGCAAAAGATCAAAgtatgtttcgaatgatactaacataacatagaa
cattttcagGAACTCCTCAAGGATAAGCCAGAGCACGTGGGAGTGAAAGT
TGGAGTTAGAACACGAGGATGCAACGGACTCTCATACACACTCGAGTACA
CAAAGTCGAAGGGAGATTCGGATGAAGAGGTGGTGCAAGATGGAGTGCGA
GTGTTCATTGAGAAGgtaagtttaaactgagttctactaactaacgagta
atatttaaattttcagAAGGCCCAACTCACACTCCTCGGAACAGAGATGG
ATTACGTCGAGGATAAGCTCTCGTCGGAGTTCGTGTTCAACAACCCAAAC
ATCAAGGGAACATGCGGATGCGGAGAGTCGTTCAACATTTGA

After applying the external magnet, zdEx12 transgenic animals displayed robust and reproducible locomotion activity, exhibiting simultaneous contractions of body muscles with apparent shrinkages of the whole body length on bacteria-fed NGM agar plates (FIG. 5B).

To quantify the effect of MAR-dependent activation on locomotion (Nagel et al., 2005; Zhang et al., 2007), we calculated the percentage of body shrinkage. This revealed shrinkages of the body length up to 6% (FIG. 5C). In contrast, there was no detectable contraction in the wild type N2 C. elegans when the external magnetic fields were applied (p<0.001, paired t-test). These results demonstrated that MAR can trigger magnet-evoked body contractions or shrinkages of C. elegans in vivo.

We next assessed whether magnet-evoked MAR could depolarize neuronal cells and cause subsequent behaviors. We made another zdEx22 transgenic C. elegans in which MAR was selectively expressed only in 6 mechanosensory neurons AVM, ALML/R, PVM and PLML/R driven by promoter mec-4 (O'Hagan et al., 2005; Zhang et al., 2007). FIG. 5D showed that MAR expression was limited to mechanosensory neurons only under the promoter of mec-4 (see also FIG. S5B). MAR triggered withdrawal behaviors in C. elegans when the magnetic field was switched on (FIG. 5E). 19 out of 22 (86%) zdEx22 transgenic animals showed robust and repeatable withdrawal behaviors under stimulation of magnetic field, in consistency with previous results from MAR-activated neurons (Nagel et al., 2005). Remarkably, we observed dramatic omega movement of the whole body of the worm after the external magnetic field was applied, indicating that unlimited accessibility of the magnetic field could activate all of the 6 mechanosensory neurons. The same result could not be obtained with optogenetics, which was limited to stimulating only a portion of the 6 mechanosensory neurons by limited light-affected penetration (Nagel et al., 2005). Occasionally, we could observe accelerations with forwarding behaviors in a few of the transgenic animals. The withdrawal behaviors could be reproducibly evoked by the external magnetic field (FIG. 5F). In contrast, those wild-type control animals did not display withdrawal or acceleration behaviors. Taken together, these results suggest that magnetogenetic control of neuronal activity by MAR could induce behavior output in vivo.

Discussion

The main discovery of our study is the neurotechnological and conceptual invention of magnetogenetics. The non-invasive magnetogenetics combines the genetic activation of neuronal activity via a magnet-dependent magnetoreceptor MAR with an external magnetic field, enabling non-invasive and wireless perturbation of neuronal activities.

Nanoparticle-Based Magnetothermal Control of Neuromodulation

Anikeeva and her colleagues (Chen et al., 2015) recently introduced a magnetothermal neuromodulation tool that involved delivering heat-sensitive capsaicin receptor TRPV1 to a particular brain area and then injecting heat-emitting nanoparticles into the same area. This two-step magnetothermal approach has intrinsic drawbacks. First, major safety issues arise from the exogenous Fe₃O₄ magnetic nanoparticles permanently incorporated into the brain and from the elevated temperature above 43° C., well exceeding physiological temperature by heat-emitting magnetic nanoparticles. Second, the diffused magnetic nanoparticles might activate other endogenous thermosensitive ion channels expressed in both peripheral and central nervous systems (Leibiger and Berggren, 2015; Temel and Jahanshahi, 2015). Third, since the resonance of magnetic nanoparticles is necessary for producing heat to open TRPV1 channels by alternating magnetic field (Chen et al., 2015), relative strong magnetic field is desired for neuronal activation (˜180 mT versus up to ˜2.5 mT in our study).

The Molecular and Cellular Mechanism of Magnetoreception

Vidal-Gadea et al. (Vidal-Gadea et al., 2015) have recently identified a pair of magnetosensory neurons from C. elegans called AFD sensory neurons that respond to geomagnetic field of the earth and support vertical migrations. It remains, however, elusive how AFD sensory neurons detect and use the earth's magnetic field to guide behaviors. Our finding demonstrates for the first time that a single gene encoding the magnetoreceptor MAR could act as a magnetic actuator for controlling neuronal activity. We hypothesize that MAR might work as a molecular biocompass in animal and play an important role in spatial navigation, migration, orientation and homing (Mouritsen and Ritz, 2005). We speculate that this novel iron-containing magnetoreceptor (MAR) might form as an iron-sulfur cluster assembly that could bind to cellular plasma membrane through either cytoskeletons or filaments (Winkhofer, 2012; Johnsen and Lohmann, 2005). After the application of the external magnetic field, the membrane tension due to the magnet-driven rotating force via MAR might cause ion channels to open, thus inducing membrane depolarization and action potential trains (Fleissner et al., 2007; Winkhofer, 2012; Wu and Dickman, 2012). We do not yet know the exact mechanism how the direction of the magnetic field and switching the magnetic field on or off affect the neuronal activity. Further insights could be obtained by studying whether the expression level of MAR, the precise alignment between the three-dimensional magnetic field stimulation and the axon-dendritical orientation of MAR-expressed neurons and/or magnetic strength might affect the direction-dependent magnetic control of neuronal activity (Mouritsen and Ritz, 2005). Further studies on MAR-interactive partners and MAR's own advanced structure might uncover the molecular mechanism for magnetogenetic control of neuronal activity.

Advantages of Magnetogenetics

Our newly invented magnetogenetics has several unique advantages over a decade-long yet still being optimized optogenetics: magnetogenetics is non-invasive, remote, penetrative, uniform, and safe. Compared to the optic fiber used in optogenetics (Fenno et al., 2011) and the electric wire assembled in deep-brain stimulation (Creed et al., 2015), there is no need for chronic surgical implantation of any invasive devices since the external magnetic fields can penetrate deeply into the intact mammalian brain or other biological systems. Although red-shifted opsins such as ReaChR (Lin et al., 2013) and Jaws (Chuong et al., 2014) permit transcranial activation or inhibition of neural activity, respectively, both ReaChR and Jaws can be effective up to only 3 mm deep in the rodent brain (Chuong et al., 2014). In the meantime, the controllable magnetic field can uniformly act on any central or peripheral nervous systems with precise genetic targeting, overcoming the effect of unevenness due to the light absorption and scattering (Häusser 2014). Furthermore, magnetogenetic stimulation within millitesla range causes no side effects like phototoxicity or thermotoxicity, making magnetogenetics much safer.

Combination of Magnetogenetics with Other Neuronal Readouts

Like all existing genetic and optogenetic activators, silencers, sensors and effectors (Adamantidis et al., 2014), this magnetoreceptor uses a single 133-amino-acid-encoded open reading frame without any co-factor for effective magnetic stimulation. By the use of neuronal cell-type-specific, sub-region-specific or sub-layer-specific promoters, delivery of this magnetoreceptor into viral and/or transgenic accessible animals will enable circuit-specific, projection-targeted and spatiotemporal mapping, manipulation, measurement and monitoring of neuronal activity in a non-invasive way. A combination of magnetogenetics with genetically encoded calcium indicators and voltage sensors (Knopfel, 2012; St-Pierre et al., 2013), multi-electrode array (Spira and Hai, 2013), functional magnetic resonance imaging (Desai et al., 2011; Lee et al., 2010) or multisite single-unit recording (Zhang et al., 2013) will allow us to record large-scale neuronal activity (Scanziani and Häusser, 2009; Häusser, 2014) and identify activity patterns corresponding to specific behavioral functions. The application of magnetogenetics will accelerate systematic and causal dissection of neural computation and coding underlying complex interconnected and interdependent brain circuit (Bargmann et al., 2014). Although our study only focuses on magnetic activation by MAR, the opposite way for magnetic inactivation from either a mutated MAR or another undiscovered magnetoreceptor by comparative genomics is feasible. Like direct optogenetic engineering (Zhang et al., 2011), the continuous molecular engineering of diverse families of magnetoreceptors will expand the magnetogenetic toolboxes.

The Application of Magnetogenetics to Translational Neuroscience

Although deep brain stimulation for treating Parkinson's disease and other neurological disorders has been proven to be effective, it uses surgically implanted metal electrodes that stimulate targeted regions without any cell-type specificity (Benabid, 2015; Creed et al., 2015; Gradinaru et al., 2009). While non-invasive transcranial magnetic stimulation (TMS) uses magnetic pulses to induce small electrical currents to stimulate a small region of the cortex (Ridding and Rothwell, 2007; Walsh and Cowey, 2000), its application for basic research and diagnostic and therapeutic use for diseases such as depression and Parkinson's disease is limited by a lack of specificity, reliability and replicability. Combined with cell-type specific promoters (Luo et al., 2008), magnetogenetics can achieve precisely targeted neuromodulation, overcome non-specificity, and have the potential to benefit therapeutic treatments for Parkinson's disease as well as other neurological and neuropsychiatric diseases.

Outlook for Magnetogenetics

In summary, non-invasive magnetic activation of neuronal activity with a magnetoreceptor makes magnetogenetics an excellent toolbox for perturbing the activity of complex neural circuitry, enabling the dissection of complex neuronal microcircuitry with cell type specificity, spatiotemporal precision, spatial uniformity and non-invasive reversibility. Combined with the genetic targeting of specific cell types and regions, magnetogenetics will accelerate our quest for reaching the ultimate goal of neuroscience: understanding how the brain computes neuronal algorithm, transforms information and generates cognition and behavior. Not only will magnetogenetics have a broad range of applications to basic and translational neuroscience, its principle of using magnetic field for non-invasive, spatiotemporal control of biological systems will also impact other fields in biological science and biomedical engineering (Etoc et al., 2015; Stanley et al., 2015) at multiple levels including genetic, epigenetic and transcriptional levels (Cong et al., 2013). Like optogenetics with progressive improvement over the past decade, we confidently envision that, with continuous research, development and optimization, a new age of magnetogenetics is coming in the near future.

### REFERENCES

1. Neuron. 2008 Mar. 13; 57(5):634-60. doi: 10.1016/j.neuron.2008.01.002. PMID: 18341986
2. http://www.nih.gov/science/brain/2025/ (US National Institutes of Health, 2014). Bargmann, C. et al. BRAIN 2025: a scientific vision. Brain Research through Advancing
3. Nature. 2013 Nov. 7; 503(7474):51-8. doi: 10.1038/nature12654. Review. PMID: 24201278
4. Annu Rev Neurosci. 2013 Jul. 8; 36:183-215. doi: 10.1146/annurev-neuro-062012-170307. Epub 2013 May 17. Review. PMID: 23682658
5. Cell. 2011 Dec. 23; 147(7):1446-57. doi: 10.1016/j.ce11.2011.12.004. PMID: 22196724
6. Neuron. 2015 May 20; 86(4):936-46. doi: 10.1016/j.neuron.2015.03.065. Epub 2015 Apr. 30. PMID: 25937170
7. Neuron. 2007 Apr. 5; 54(1):35-49. PMID: 17408576
8. Neuron. 2006 Oct. 5; 52(1):197-204. Review. PMID: 17015236
9. Proc Natl Acad Sci USA. 1992 Jun. 15; 89(12):5675-9. PMID: 1608978
10. Nature. 2008 Jun. 12; 453(7197):869-78. doi: 10.1038/nature06976. Review. PMID: 18548064
11. Science. 2009 Apr. 17; 324(5925):354-9. doi: 10.1126/science.1167093. Epub 2009 Mar. 19. PMID: 19299587
12. Nat Rev Neurosci. 2007 August; 8(8):623-35. PMID: 17637800
13. Nat Neurosci. 2005 September; 8(9):1263-8. Epub 2005 Aug. 14. PMID:16116447
14. Proc Natl Acad Sci USA. 2005 Dec. 6; 102(49):17816-21. Epub 2005 Nov. 23. PMID: 16306259
15. Neuron. 2006 Apr. 6; 50(1):23-33. PMID: 16600853
16. Nat Methods. 2014 October; 11(10):1012-4. doi: 10.1038/nmeth.3111. PMID: 25264778
17. Neuron. 2015 Apr. 8; 86(1):106-39. doi: 10.1016/j.neuron.2015.03.034. Review. PMID: 25856490
18. N Engl J Med. 2012 Oct. 18; 367(16):1529-38. doi: 10.1056/NEJMct1208070. PMID: 23075179
19. Metallomics. 2012 August; 4(9):928-36. doi: 10.1039/c2mt20065a. PMID: 22885802
20. Biochim Biophys Acta. 2004 Aug. 2; 1700(2):179-88. PMID: 15262227
21. Nature. 2013 Jul. 18; 499(7458):295-300. doi: 10.1038/nature12354. PMID: 23868258
22. Nat Methods. 2009 December; 6(12):875-81. doi: 10.1038/nmeth.1398. Epub 2009 Nov.
8. PMID: 19898485
23. Curr Opin Neurobiol. 2005 August; 15(4):406-14. Review. PMID: 16006116
24. Neuron. 2007 Feb. 15; 53(4):549-62. PMID: 17296556
25. J Cell Biol. 2000 Sep. 18; 150(6):1423-34. PMID: 10995446
26. Science. 2012 May 25; 336(6084):991-2. doi: 10.1126/science.1223786. No abstract available. PMID: 22628645
27. Nature. 2004 Nov. 25; 432(7016):508-11. PMID: 15565156
28. Brain Res Bull. 1990 November; 25(5):735-40. PMID: 2289162
29. Curr Protoc Neurosci. 2001 May; Chapter 6:Unit 6.6. doi: 10.1002/0471142301.ns0606s00. PMID: 18428516
30. Curr Biol. 2005 Dec. 20; 15(24):2279-84. PMID: 16360690
31. Genetics. 1993 October; 135(2):385-404. PMID: 8244003
32. Biol Cell. 2013 June; 105(6):235-50. doi: 10.1111/boc.201200069. Epub 2013 Apr. 26. PMID: 23458457
33. Cell. 2009 Oct. 30; 139(3):623-33. doi: 10.1016/j.ce11.2009.08.044. PMID: 19879847
34. Nature. 2007 Apr. 5; 446(7136):633-9. PMID: 17410168
35. PLoS One. 2007 Mar. 21; 2(3):e299. PMID: 17375185
36. Nat Neurosci. 2005 January; 8(1):43-50. Epub 2004 Dec. 5. PMID: 15580270
37. Science. 2015 Mar. 27; 347(6229):1477-80. doi: 10.1126/science.1261821. Epub 2015 Mar. 12. PMID: 25765068
38. Nat Med. 2015 January; 21(1):14-6. doi: 10.1038/nm.3782. PMID: 25569545
39. Science. 2015 Mar. 27; 347(6229):1418-9. doi: 10.1126/science.aaa9610. No abstract available. PMID: 25814569
40. Elife. 2015 Jun. 17; 4. doi: 10.7554/eLife.07493. PMID: 26083711
41. Nat Rev Neurosci. 2005 September; 6(9):703-12. PMID: 16100517
42. Science. 2012 May 25; 336(6084):1054-7. doi: 10.1126/science.1216567. Epub 2012 Apr. 26. PMID: 22539554
43. Naturwissenschaften. 2007 August; 94(8):631-42. Epub 2007 Mar. 15. PMID: 17361399
44. Annu Rev Neurosci. 2011; 34:389-412. doi: 10.1146/annurev-neuro-061010-113817. PMID: 21692661
45. Science. 2015 Feb. 6; 347(6222):659-64. doi: 10.1126/science.1260776. PMID: 25657248
46. Nat Neurosci. 2013 October; 16(10):1499-508. doi: 10.1038/nn.3502. Epub 2013 Sep. 1. PMID:23995068
47. Nat Neurosci. 2014 August; 17(8):1123-9. doi: 10.1038/nn.3752. Epub 2014 Jul. 6. PMID: 24997763
48. Cold Spring Harb Protoc. 2014 Aug. 1; 2014(8):815-22. doi: 10.1101/pdb.top083329. PMID: 25086025
49. Nat Rev Neurosci. 2012 October; 13(10):687-700. doi: 10.1038/nrn3293. Epub 2012 Aug. 30. Review. PMID: 22931891
50. Nat Neurosci. 2014 June; 17(6):884-9. doi: 10.1038/nn.3709. Epub 2014 Apr. 22. PMID: 24755780
51. Nat Nanotechnol. 2013 February; 8(2):83-94. doi: 10.1038/nnano.2012.265. Review. PMID: 23380931
52. Nature. 2010 Jun. 10; 465(7299):788-92. doi: 10.1038/nature09108. PMID: 20473285
53. J Neurophysiol. 2011 March; 105(3):1393-405. doi: 10.1152/jn.00828.2010. Epub 2010 Dec. 15. PMID: 21160013
54. Nature. 2009 Oct. 15; 461(7266):930-9. doi: 10.1038/nature08540. Review. PMID: 25264778
55. Science. 2013 Feb. 15; 339(6121):819-23. doi: 10.1126/science.1231143. Epub 2013 Jan. 3.
56. Nature. 2015 Mar. 19; 519(7543):299-300. doi: 10.1038/519299a. PMID: 25788094
57. Nat Rev Neurosci. 2007 July; 8(7):559-67. Review. PMID: 17565358
58. Nat Rev Neurosci. 2000 October; 1(1):73-9. Review. PMID:11252771
59. Nano Lett. 2015 May 13; 15(5):3487-94. doi: 10.1021/acs.nanolett.5b00851. Epub 2015 Apr. 28. PMID: 25895433
60. Nat Med. 2015 January; 21(1):92-8. doi: 10.1038/nm.3730. Epub 2014 Dec. 15.

Erratum in: Nat Med. 2015 May; 21(5):537. PMID: 25501906
61. PLoS Genet. 2009 August; 5(8):e1000604. doi: 10.1371/journal.pgen.1000604. Epub 2009 Aug. 14. PMID: 19680447
62. J Neurosci. 2011 Mar. 30; 31(13):4978-90. doi: 10.1523/JNEUROSCI.2672-10.2011. PMID: 21451036

1. A method of modulating the activity of a cell, comprising the steps of delivering a MAR gene into said cell and providing a magnetic stimulation to said cell.

2. (canceled)

3. The method of claim 1, wherein the cell is treated in vitro.

4. The method of claim 1, wherein the cell is in a subject.

5. The method of claim 4, wherein the subject is a human.

6. The method of claim 5, wherein the MAR gene is codon optimized for expression in a human.

7. The method of claim 1, wherein the sequence of the MAR gene is selected from the group consisting of SEQ ID NOs:1-11.

8. The method of claim 1, wherein the MAR gene is delivered to a target location via a vector comprising a cell type specific promoter or region specific promoter.

9. The method of claim 8, wherein the vector comprises a lentivirus, a retrovirus or adeno-associated virus or a plasmid.

10. A method of treating a subject, comprising the steps of delivering a MAR gene to a target region in the subject and providing a magnetic stimulation to the region.

11. (canceled)

12. (canceled)

13. The method of claim 10, wherein the subject has a disease or injury selected from the group consisting of spinal cord injury, neurodegenerative diseases, retina-degenerative diseases, cardiac diseases, Sjögren's syndrome and addiction.

14. The method of claim 10, wherein the MAR gene is targeted to one or more diseased regions by a vector which comprises a cell type specific promoter or region specific promoter.

15. The method of claim 10, wherein the MAR gene is delivered by implanting a MAR-expressing cell into said subject.

16. (canceled)

17. The method of claim 13, wherein the neurodegenerative disease is selected from the group consisting of Alzheimer's disease, Parkinson's disease, Prion disease, Motor neuron diseases, Huntington's disease, Spinocerebellar ataxia, and Spinal muscular atrophy.

18-34. (canceled)

35. The method of claim 10, wherein the MAR gene is delivered by implanting a MAR-expressing cell into said subject.

36-43. (canceled)

44. A pharmaceutical composition for treating a subject, comprising:
a vector comprising a MAR gene, or a MAR-expressing cell; and
a pharmaceutically acceptable carrier.

45-52. (canceled)


SEQUENCE LISTING (11 sequences)

SEQ ID NO: 1 - DNA, 393 bp, fruit fly
atggcgacacgtgtggtggcaacggcgacagtgcgggcggtgaaaggccggaagttaatcccgacgcgggccgctctgactctgacacccgcggcggtgctacgcatcaagacgcttctgcaggacaagccggacatggttggcctaaaggtgggcgtacggcagcgaggatgcaatggtctgtcctacacgctggactatgccagccaaaaagacaagttggatgaggaggtggtccaggatggcgtcaaggtcttcatcgacaagaaagcgcagttgtcgctgctgggtaccgagatggactttgtggaatcgaagctgtccagcgagttcgtgtttaacaatccgaacattaagggcacatgcggctgcggcgaatcgttcagcatgtaa

SEQ ID NO: 2 - DNA, 396 bp, butterfly
atgtctactaaaactatagcaagtgcaactgttagggcagtaaaaaagcgtctgctaccatccagagctgctctagttttgacttcttcagccgtaaataaagttaaggaaataatggccaaggaagaaggcaagggttatataggattgaaagttggtgtgcggcaaagaggttgcaatggattgtcatataccttagattatgcaacatcaaaagggaaacttgacgaagaagtaaaacaggatggagtcactataattattgacaaaaaagcacagttgaccttgttgggtactgaaatggattttgtcgaagataagctgtcagcggaatttgtgtttaacaatccgaatataaaaggcacttgtggatgtggagaatctttcagtatataa

SEQ ID NO: 3 - DNA, 399 bp, pigeon
atggcttcttctgcttcttctgttgttcgtgctaccgttcgtgctgtttctaaacgtaaaatccaggctacccgtgctgctctgaccctgaccccgtctgctgttcagaaaatcaaagaactgctgaaagacaaaccggaacacgttggtgttaaagttggtgttcgtacccgtggttgcaacggtctgtcttacaccctggaatacaccaaatctaaaggtgactctgacgaagaagttgttcaggacggtgttcgtgttttcatcgaaaaaaaagctcagctgaccctgctgggtactgaaatggactacgttgaagacaaactgtcttctgaattcgttttcaacaacccgaacatcaaaggtacttgcggttgcggtgaatctttcaacatctaa

SEQ ID NO: 4 - DNA, 390 bp, mouse
atgtcggcgtcgttggtccgcgccaccgtgcgggctgtgagcaagagaaaactgcagcccacgcgggcggccctcacactgaccccctctgctgtaaacaagataaaacaacttcttaaagacaaacctgagcatgtgggtctgaaagttggcgtgcgaaccaggggctgtaatggcctctcttacagcctggagtacacaaagacaaaaggagattctgatgaagaagttattcaagatggagtccgagtgttcatcgagaagaaagcacagctaaccctgttaggaacagagatggactatgtggaagacaaactgtccagtgagtttgtgttcaataaccccaacatcaagggaacctgtggctgcggtgagagctttcacgtgtga

SEQ ID NO: 5 - DNA, 390 bp, rat
atgtcggcgtcgttggtccgcgccaccgtgcgggccgtgagcaagagaaaactgcaacccacgcgggcggcgctcacgctgaccccctctgctgtgaacaagataaaacaacttcttaaagacaagcctgagcatgtgggtctgaaagtgggtgtgcggaccaggggctgtaacggcctctcttacagcctggagtatacaaagacaaaaggagatgctgatgaagaagttattcaagacggagtccgagtgttcatcgagaagaaagcccagctaaccctgttaggcacagagatggactatgtggaagacaaactgtccagtgagtttgtgttcaacaaccccaacatcaagggaacctgtggctgcggtgaaagctttaacgtctga

SEQ ID NO: 6 - DNA, 390 bp, Homo sapiens
atgtcggcttccttagtccgggcaactgtccgggctgtgagcaagaggaagctgcagcccacccgggcagccctcaccctgacaccttcagcagtaaacaagataaaacaacttcttaaagataagcctgagcatgtaggtgtaaaagttggtgtccgaaccaggggctgtaatggcctttcttatactctagaatatacaaagacaaaaggagattctgatgaagaagttattcaagatggagtcagagtattcatcgaaaagaaagcacagctaacacttttaggaacagaaatggactatgttgaagacaaattatccagtgagtttgtgttcaataacccaaacatcaaagggacttgtggctgtggagaaagctttaatatttga

SEQ ID NO: 7 - DNA, 399 bp, Artificial Sequence
  synthetic codon-optimized pigeon MAR gene for expression in mouse
atggcctctagcgcctctagcgtcgtgagagctaccgtgagagccgtgtccaagaggaagatccaggccacaagagccgctctgacactgacccctagcgccgtgcagaagatcaaggagctgctgaaggacaagcccgaacacgtgggagtgaaagtgggcgtgcgaaccagaggttgcaacggcctgagctacaccctggagtacaccaagagcaagggcgacagcgacgaggaagtggtgcaggacggagtgcgagtgttcatcgagaagaaggcccagctgacactgctgggaaccgagatggactacgtggaggacaagctgagcagcgagttcgtgttcaacaaccccaacatcaagggcacttgcggctgcggcgagtcttttaacatctag

SEQ ID NO: 8 - DNA, 399 bp, Artificial Sequence
  synthetic codon-optimized pigeon MAR gene for expression in rat
atggcctctagcgcctctagcgtcgtgagagctaccgtgagagccgtgtccaagaggaagatccaggccacaagagccgctctgacactgacccctagcgccgtgcagaagatcaaggagctgctgaaggacaagcccgaacacgtgggagtgaaagtgggcgtgcgaaccagaggttgcaacggcctgagctacaccctggagtacaccaagagcaagggcgacagcgacgaggaagtggtgcaggacggagtgcgagtgttcatcgagaagaaggcccagctgacactgctgggaaccgagatggactacgtggaggacaagctgagcagcgagttcgtgttcaacaaccccaacatcaagggcacctgcggatgcggcgagtcttttaacatctag

SEQ ID NO: 9 - DNA, 399 bp, Artificial Sequence
  synthetic codon-optimized pigeon MAR gene for expression in marmoset
atggctagcagcgctagcagcgtggtgagggctaccgtgagggccgtgtccaagaggaagatccaggctaccagggccgctctgactctgactccaagcgccgtgcagaagatcaaggagctgctgaaggacaagcccgaacacgtgggagtgaaggtgggagtgaggaccaggggttgtaacggcctgagctacaccctggagtacaccaagagcaagggcgacagcgacgaggaagtggtgcaggacggagtgagggtgttcatcgagaagaaggctcagctgaccctgctgggaaccgagatggactacgtggaggacaagctgagcagcgagttcgtgttcaacaaccccaacatcaagggcacttgcggttgcggcgagagctttaacatctaa

SEQ ID NO: 10 - DNA, 399 bp, Artificial Sequence
  synthetic codon-optimized pigeon MAR gene for expression in human
atggctagcagcgcctctagcgtcgtgagagctacagtgcgggccgtgtccaagagaaagatccaggccacaagagccgctctgacactgacccctagcgccgtgcagaagatcaaggagctgctgaaggacaagcccgaacacgtgggagtgaaagtgggcgtgaggaccagaggttgcaacggcctgagctacaccctggagtacaccaagagcaagggcgacagcgacgaggaagtggtgcaggacggagtgcgagtgttcatcgagaagaaggcccagctgacactgctgggaaccgagatggactacgtggaggacaagctgagcagcgagttcgtgttcaacaaccccaacatcaagggcacttgcggctgcggcgagtcttttaacatctag

SEQ ID NO: 11 - DNA, 492 bp, Artificial Sequence
  synthetic MAR gene used in C. elegans
atggcttcgtcggcctcatcagttgttagagctacagttcgagccgtgtcgaagcgaaagattcaagctacacgagctgccctcacactcacaccatcagctgtgcaaaagatcaaagtatgtttcgaatgatactaacataacatagaacattttcaggaactcctcaaggataagccagagcacgtgggagtgaaagttggagttagaacacgaggatgcaacggactctcatacacactcgagtacacaaagtcgaagggagattcggatgaagaggtggtgcaagatggagtgcgagtgttcattgagaaggtaagtttaaactgagttctactaactaacgagtaatatttaaattttcagaaggcccaactcacactcctcggaacagagatggattacgtcgaggataagctctcgtcggagttcgtgttcaacaacccaaacatcaagggaacatgcggatgcggagagtcgttcaacatttga